(19) United States
(12) Patent Application Publication
(10) Pub. No.: US 2023/0140384 A1
(43) Pub. Date: May 4, 2023

(54) ANTIBODIES TO CD40 WITH ENHANCED AGONIST ACTIVITY

(71) Applicant: BRISTOL-MYERS SQUIBB COMPANY, Princeton, NJ (US)

(72) Inventors: Arvind RAJPAL, San Francisco, CA (US); Aaron Paul YAMNIUK, Vancouver (CA); Pavel STROP, San Mateo, CA (US); Bryan C. BARNHART, Vancouver (CA); Feng WANG, Belmont, CA (US)

(21) Appl. No.: 17/905,870

(22) PCT Filed: Mar. 8, 2021
(86) PCT No.: PCT/US2021/021339

§ 371 Date: Sep. 8, 2022

Related U.S. Application Data
(60) Provisional application No. 62/987,114, filed on Mar. 9, 2020.

(51) Int. Cl. C07K 16/28; C12N 15/63; A61P 37/04

(52) U.S. Cl. CPC C07K 16/2878; C12N 15/63; A61P 37/04; C07K 2317/565

(57) ABSTRACT

Provided herein are agonist antibodies that bind to human CD40 with enhanced agonist activity. Such antibodies comprise Fc regions with amino acid substitutions that enhance the agonist activity of the antibody compared with a similar IgG1 antibody. Such substitutions include sequence variants in the IgG2 hinge region and sequence variants that enhance hexamerization of the antibodies. The invention also provides methods of treatment of cancer or chronic infection by administering the antibodies of the invention to a subject in need thereof.

### CROSS REFERENCE TO RELATED APPLICATIONS

This application claims priority to U.S. Provisional Application No. 62/987,114, filed Mar. 9, 2020, the disclosure of which is incorporated herein by reference.

### SEQUENCE LISTING

The Sequence Listing filed electronically herewith is also hereby incorporated by reference in its entirety (File Name: 20210215_SEQL_13408WOPCT_GB.txt; Date Created: 15 Feb. 2021; File Size: 413 KB).

### BACKGROUND

Research has revealed that human cancers and chronic infections may be treated with agents that modulate the patient's immune response to malignant or infected cells. See, e.g., Reck & Paz-Ares (2015) Semin. Oncol. 42:402. Agonistic anti-CD40 antibodies, such as CP-870893 and dacetuzumab (SGN-40) have been tried for treating cancer based on the belief that they may enhance such an immune response. See, e.g., Kirkwood et al. (2012) CA Cancer J. Clin. 62:309; Vonderheide & Glennie (2013) Clin. Cancer Res. 19:1035. Other experiments in mice have revealed that anti-CD40 antibodies with enhanced specificity for the inhibitory Fc receptor FcγRIIb have increased anti-tumor efficacy. See, e.g., WO 2012/087928; Li & Ravetch (2011) Science 333:1030; Li & Ravetch (2012) Proc. Nat'l Acad. Sci (USA) 109:10966; Wilson et al. (2011) Cancer Cell 19:101; White et al. (2011) J. Immunol. 187:1754; WO 2017/004006.

The need exists for improved agonistic anti-human CD40 antibodies for treatment of cancer and chronic infections in human subjects. Such antibodies will preferably have enhanced agonist activity compared with antibodies having human IgG1 constant regions.

### SUMMARY OF THE INVENTION

Provided herein are isolated humanized murine monoclonal antibodies that specifically bind to human CD40 (the mature sequence of SEQ ID NO: 1) having modified constant regions that increase agonist activity. In certain embodiments, the anti-human CD40 antibodies of the present invention comprise an antibody selected from the group consisting of mAb 12D6, 5F11, 8E8, 5G7 and 19G3 having modified constant regions that increase agonist activity.

In one aspect, the present invention provides isolated monoclonal antibodies, or antigen binding portions thereof, that bind to human CD40, comprising a modified heavy chain Fc region that comprises mutations that enhance hexamerization of the antibodies, such as E345K or E345R, optionally in combination with one or both of E430G and S440Y, or E430G alone. Such hexamerization mutants may be introduced into an IgG1 heavy chain constant region, or alternatively in an IgG2 heavy chain constant region.

In another aspect, the present invention provides isolated monoclonal antibodies, or antigen binding portions thereof, that bind to human CD40, comprising a modified heavy chain Fc region that comprises an IgG2 hinge and at least one of CH1, CH2 and CH3 that is not of an IgG2 isotype. In one such embodiment, the antibody comprises a wildtype human IgG2 hinge (SEQ ID NO: 77) or a variant thereof comprising one or more mutations selected from the group consisting of C219S, C220S, C226S and C229S. In some embodiments, the antibody comprises a hybrid constant region comprising an IgG2 CH1 domain and upper and middle hinge regions, and an IgG1 lower hinge region and CH2 and CH3 domains.

In some embodiments the IgG2 portion of the constant region is modified to replace a cysteine residue with a serine residue to minimize disulfide shuffling, which can lead to undesired heterogeneity in an antibody preparation. In one embodiment the IgG2 CH1 domain comprises a C131S mutation (IgG2.5; SEQ ID NO: 89), and in another embodiment the IgG2 upper hinge region comprises a C219S mutation (IgG2.3; SEQ ID NO: 86).

In some embodiments the antibodies of the present invention are further modified to reduce unwanted effector function. In some such embodiments the lower hinge region includes the three mutations L234A, L235E and G237A, referred to as IgG1.3f (SEQ ID NO: 155). In other such embodiments, the antibodies comprise the P238K or D265A mutation, optionally further comprising the L235E mutation, and further optionally including the K322A mutation (to reduce complement binding). Fc receptor binding experiments are described at Example 1 and results are provided at Table 8.

In various specific embodiments, the agonist anti-CD40 antibodies with enhanced agonist activity of the present invention form hexamers, and comprise a variant IgG1 heavy chain constant region selected from the group consisting of SEQ ID NOs: 171-177, such as SEQ ID NOs: 174-175, or a variant IgG2 heavy chain constant region selected from the group consisting of SEQ ID NOs: 178-185. In some embodiments, the antibody that forms hexamers exhibits reduced or eliminated effector function, such as CDC, when compared with an otherwise identical antibody having a wildtype IgG1f constant region (SEQ ID NOs: 44 and 85), e.g. an antibody comprising a variant IgG1 heavy chain constant region selected from the group consisting of SEQ ID NOs: 173-177, 184 and 185.

In other specific embodiments, the agonist anti-CD40 antibodies with enhanced agonist activity of the present invention comprise IgG2 hinge domains, and comprise a hybrid IgG2/IgG1 heavy chain constant region selected from the group consisting of SEQ ID NOs: 159-170, such as SEQ ID NOs: 159-161 and 165-167.

In various other aspects, the anti-CD40 antibody with enhanced agonist activity of the present invention comprises an antigen binding domain are structurally or functionally related to the specific agonist anti-CD40 antibodies disclosed herein by sequence (mAbs 12D6, 5F11, 8E8, 5G7 and 19G3—collectively referred to as the disclosed antibodies). Such aspects include antibodies comprising a heavy chain constant region selected from the group consisting of SEQ ID NOs: 159-185, further comprising an antigen binding domain that competes with one or more of the disclosed antibodies for binding to human CD40, that bind to the same epitope as one of the disclosed antibodies, or are derived from the same murine germline sequences as the one of the disclosed antibodies. In one specific embodiment, the antigen binding domain of the antibody of the present invention reduces the binding of the disclosed antibody to bind to human CD40 (SEQ ID NO:1) in a competition ELISA by at least 20% when used at an equimolar concentration with the disclosed antibody. In another specific embodiment, the antigen binding domain of the antibody of the present invention binds to an epitope comprising or consisting of the epitope bound by mAb 12D6 (residues 11-35 of SEQ ID NO:1), the epitope bound by mAb 5G7 (residues 21-35 of SEQ ID NO:1), or the epitope bound by mAb 5F11 (residues 58-66 of SEQ ID NO:1). In a further specific embodiment, the antigen binding domain of the antibody of the present invention is derived from heavy chain V region germline VH1-39_01 and J region germline IGHJ4 and light chain V region germline VK1-110_01 and J region germline IGKJ1 (12D6); heavy chain V region germline VH1-4_02 and J region germline IGHJ3 and light chain V region germline VK3-5_01 and J region germline IGKJ5 (5F11); heavy chain V region germline VH1-80_01 and J region germline IGHJ2 and light chain V region germline VK1-110_01 and J region germline IGKJ2 (8E8); heavy chain V region germline VH1-18_01 and J region germline IGHJ4 and light chain V region germline VK10-96_01 and J region germline IGKJ2 (5G7); or heavy chain V region germline VH5-9-4_01 and J region germline IGHJ3 and light chain V region germline VK1-117_01 and J region germline IGKJ2 (19G3).

In various embodiments the antibody of the present invention comprises a heavy chain and a light chain, wherein the heavy chain comprises a constant region selected from the group consisting of SEQ ID NOs: 159-185, and also comprises CDRH1, CDRH2 and CDRH3 sequences and the light chain comprises CDRL1, CDRL2 and CDRL3 sequences selected from the group consisting of: the CDRs of antibody 12D6-03 wherein CDRH1, CDRH2 and CDRH3 comprise residues 31-35, 50-66 and 99-108, respectively, of SEQ ID NO:5 and CDRL1, CDRL2 and CDRL3 comprise residues 24-39, 55-61 and 94-102, respectively, of SEQ ID NO:6; the CDRs of antibody 12D6-22 wherein CDRH1, CDRH2 and CDRH3 comprise residues 31-35, 50-66 and 99-108, respectively, of SEQ ID NO:7 and CDRL1, CDRL2 and CDRL3 comprise residues 24-39, 55-61 and 94-102, respectively, of SEQ ID NO:8; the CDRs of antibody 12D6-23 wherein CDRH1, CDRH2 and CDRH3 comprise residues 31-35, 50-66 and 99-108, respectively, of SEQ ID NO:9 and CDRL1, CDRL2 and CDRL3 comprise residues 24-39, 55-61 and 94-102, respectively, of SEQ ID NO:10; the CDRs of antibody 12D6-24 wherein CDRH1, CDRH2 and CDRH3 comprise residues 31-35, 50-66 and 99-108, respectively, of SEQ ID NO:11 and CDRL1, CDRL2 and CDRL3 comprise residues 24-39, 55-61 and 94-102, respectively, of SEQ ID NO:8; the CDRs of antibody 5F11-17 wherein CDRH1, CDRH2 and CDRH3 comprise residues 31-35, 50-66 and 99-106, respectively, of SEQ ID NO:14 and CDRL1, CDRL2 and CDRL3 comprise residues 24-38, 54-60 and 93-101, respectively, of SEQ ID NO:15; the CDRs of antibody 5F11-23 wherein CDRH1, CDRH2 and CDRH3 comprise residues 31-35, 50-66 and 99-106, respectively, of SEQ ID NO:16 and CDRL1, CDRL2 and CDRL3 comprise residues 24-38, 54-60 and 93-101, respectively, of SEQ ID NO:17; the CDRs of antibody 5F11-45 wherein CDRH1, CDRH2 and CDRH3 comprise residues 31-35, 50-66 and 99-106, respectively, of SEQ ID NO:18 and CDRL1, CDRL2 and CDRL3 comprise residues 24-38, 54-60 and 93-101, respectively, of SEQ ID NO:19; the CDRs of antibody 8E8-56 wherein CDRH1, CDRH2 and CDRH3 comprise residues 31-35, 50-66 and 99-111, respectively, of SEQ ID NO:22 and CDRL1, CDRL2 and CDRL3 comprise residues 24-39, 55-61 and 94-102, respectively, of SEQ ID NO:23; the CDRs of antibody 8E8-62 wherein CDRH1, CDRH2 and CDRH3 comprise residues 31-35, 50-66 and 99-111, respectively, of SEQ ID NO:24 and CDRL1, CDRL2 and CDRL3 comprise residues 24-39, 55-61 and 94-102, respectively, of SEQ ID NO:25; the CDRs of antibody 8E8-67 wherein CDRH1, CDRH2 and CDRH3 comprise residues 31-35, 50-66 and 99-111, respectively, of SEQ ID NO:26 and CDRL1, CDRL2 and CDRL3 comprise residues 24-39, 55-61 and 94-102, respectively, of SEQ ID NO:27; the CDRs of antibody 8E8-70 wherein CDRH1, CDRH2 and CDRH3 comprise residues 31-35, 50-66 and 99-111, respectively, of SEQ ID NO:28 and CDRL1, CDRL2 and CDRL3 comprise residues 24-39, 55-61 and 94-102, respectively, of SEQ ID NO:29; the CDRs of antibody 8E8-71 wherein CDRH1, CDRH2 and CDRH3 comprise residues 31-35, 50-66 and 99-111, respectively, of SEQ ID NO:30 and CDRL1, CDRL2 and CDRL3 comprise residues 24-39, 55-61 and 94-102, respectively, of SEQ ID NO:31; the CDRs of antibody 5G7-22 wherein CDRH1, CDRH2 and CDRH3 comprise residues 31-35, 50-66 and 99-102, respectively, of SEQ ID NO:34 and CDRL1, CDRL2 and CDRL3 comprise residues 24-34, 50-56 and 89-97, respectively, of SEQ ID NO:35; the CDRs of antibody 5G7-25 wherein CDRH1, CDRH2 and CDRH3 comprise residues 31-35, 50-66 and 99-102, respectively, of SEQ ID NO:36 and CDRL1, CDRL2 and CDRL3 comprise residues 24-34, 50-56 and 89-97, respectively, of SEQ ID NO:37; the CDRs of antibody 19G3-11 wherein CDRH1, CDRH2 and CDRH3 comprise residues 31-35, 50-66 and 99-101, respectively, of SEQ ID NO:40 and CDRL1, CDRL2 and CDRL3 comprise residues 24-39, 55-61 and 94-102, respectively, of SEQ ID NO:41; and the CDRs of antibody 19G3-22 wherein CDRH1, CDRH2 and CDRH3 comprise residues 31-35, 50-66 and 99-101, respectively, of SEQ ID NO:42 and CDRL1, CDRL2 and CDRL3 comprise residues 24-39, 55-61 and 94-102, respectively, of SEQ ID NO:43.

In various embodiments the antibody of the present invention comprises a heavy chain comprising a variable domain selected from the group consisting of 12D6 (residues 1-119 of SEQ ID NO: 3), 5F11 (residues 1-117 of SEQ ID NO: 12), 8E8 (residues 1-122 of SEQ ID NO: 21), 5G7 (residues 1-113 of SEQ ID NO: 32) and 19G3 (residues 1-112 of SEQ ID NO: 38), with a heavy chain constant region comprising selected from the group consisting of SEQ ID NOs: 159-185.

In some embodiments the antibody comprises specific heavy chain variable domains and light chain variable domains selected from the group consisting of 12D6-03 (residues 1-119 and 1-112 of SEQ ID NO:5 and SEQ ID NO:6, respectively), 12D6-22 (residues 1-119 and 1-112 of SEQ ID NO:7 and SEQ ID NO:8, respectively), 12D6-23 (residues 1-119 and 1-112 of SEQ ID NO:9 and SEQ ID NO:10, respectively), 12D6-24 (residues 1-119 and 1-112 of SEQ ID NO:11 and SEQ ID NO:8, respectively), 5F11-17 (residues 1-117 and 1-111 of SEQ ID NO:14 and SEQ ID NO:15, respectively), 5F11-23 (residues 1-117 and 1-111 of SEQ ID NO:16 and SEQ ID NO:17, respectively), 5F11-45 (residues 1-117 and 1-111 of SEQ ID NO:18 and SEQ ID NO:19), 8E8-56 (residues 1-122 and 1-112 of SEQ ID NO:22 and SEQ ID NO:23, respectively), 8E8-62 (residues 1-122 and 1-112 of SEQ ID NO:24 and SEQ ID NO:25, respectively), 8E8-67 (residues 1-122 and 1-112 of SEQ ID NO:26 and SEQ ID NO:27, respectively), 8E8-70 (residues 1-122 and 1-112 of SEQ ID NO:28 and SEQ ID NO:29), 8E8-71 (residues 1-122 and 1-112 of SEQ ID NO:30 and SEQ ID NO:31, respectively), 5G7-22 (residues 1-113 and 1-107 of SEQ ID NO:34 and SEQ ID NO:35, respectively), 5G7-25 (residues 1-113 and 1-107 of SEQ ID NO:36 and SEQ ID NO:37, respectively), 19G3-11 (residues 1-112 and 1-112 of SEQ ID NO:40 and SEQ ID NO:41, respectively), and 9G3-22 (residues 1-112 and 1-112 of SEQ ID NO:42 and SEQ ID NO:43, respectively), and further comprises a heavy chain constant region comprising selected from the group consisting of SEQ ID NOs: 159-185. Any of these antibodies may further comprise the light chain kappa constant region of SEQ ID NO: 45.

In some embodiments the antibody comprises heavy chains comprising specific heavy chain variable domains selected from the group consisting of 12D6-03 (residues 1-119 of SEQ ID NO:5), 12D6-22 (residues 1-119 of SEQ ID NO:7), 12D6-23 (residues 1-119 of SEQ ID NO:9), 12D6-24 (residues 1-119 of SEQ ID NO:11), 5F11-17 (residues 1-117 of SEQ ID NO:14), 5F11-23 (residues 1-117 of SEQ ID NO:16), 5F11-45 (residues 1-117 of SEQ ID NO:18), 8E8-56 (residues 1-122 of SEQ ID NO:22), 8E8-62 (residues 1-122 of SEQ ID NO:24), 8E8-67 (residues 1-122 of SEQ ID NO:26), 8E8-70 (residues 1-122 of SEQ ID NO:28), 8E8-71 (residues 1-122 of SEQ ID NO:30), 5G7-22 (residues 1-113 of SEQ ID NO:34), 5G7-25 (residues 1-113 of SEQ ID NO:36), 19G3-11 (residues 1-112 of SEQ ID NO:40), and 9G3-22 (residues 1-112 of SEQ ID NO:42), and further comprises a heavy chain constant region comprising selected from the group consisting of SEQ ID NOs: 159-185. Any of these antibodies may further comprise the light chain kappa constant region of SEQ ID NO: 45.

In some embodiments the anti-huCD40 antibody of the present invention comprises one or more heavy chains and one or more light chains, such as two heavy chains and two light chains.

The present invention further provides nucleic acids encoding the heavy and/or light chain variable regions, of the anti-CD40 antibodies of the present invention, expression vectors comprising the nucleic acid molecules, cells transformed with the expression vectors, and methods of producing the antibodies by expressing the antibodies from cells transformed with the expression vectors and recovering the antibody.

The present invention also provides pharmaceutical compositions comprising anti-huCD40 antibodies of the present invention and a carrier.

The present invention provides a method of enhancing an immune response in a subject comprising administering an effective amount of an anti-huCD40 antibody of the present invention to the subject such that an immune response in the subject is enhanced. In certain embodiments, the subject has a tumor and an immune response against the tumor is enhanced. In another embodiment, the subject has a viral infection, e.g. a chronic viral infection, and an anti-viral immune response is enhanced.

The present invention also provides a method of inhibiting the growth of tumors in a subject comprising administering to the subject an anti-huCD40 antibody of the present invention such that growth of the tumor is inhibited.

The present invention further provides a method of treating cancer, e.g., by immunotherapy, comprising administering to a subject in need thereof a therapeutically effective amount an anti-huCD40 antibody of the present invention, e.g. as a pharmaceutical composition, thereby treating the cancer. In certain embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In certain embodiments, the cancer is a metastatic cancer, refractory cancer, or recurrent cancer.

The present invention further provides a method of treating a chronic viral infection comprising administering to a subject in need thereof a therapeutically effective amount an anti-huCD40 antibody of the present invention, e.g. as a pharmaceutical composition, thereby treating the chronic viral infection.

In certain embodiments, the methods of modulating immune function and methods of treatment described herein comprise administering an anti-huCD40 antibody of the present invention in combination with, or as a bispecific reagent with, one or more additional therapeutics, for example, an anti-PD1 antibody, an anti-PD-L1 antibody, an anti-LAG3 antibody, an anti-GITR antibody, an anti-OX40 antibody, an anti-CD73 antibody, an anti-TIGIT antibody, an anti-CD137 antibody, an anti-CD27 antibody, an anti-CSF-1R antibody, an anti-CTLA-4 antibody, a TLR agonist, or a small molecule antagonist of IDO or TGFβ. In specific embodiments, anti-huCD40 therapy is combined with anti-PD1 and/or anti-PD-L1 therapy, e.g. treatment with an antibody or antigen binding fragment thereof that binds to human PD1 or an antibody or antigen binding fragment thereof that binds to human PD-L1.

### BRIEF DESCRIPTION OF THE DRAWINGS

FIG. 1 shows the sequence of human IgG1f constant region (SEQ ID NO: 44) renumbered 117-446 to better illustrate the heavy chain constant region sequence variants disclosed herein. Residues subject to variation in the hexamer embodiments are in bold, with the altered amino acids provided in bold below the residue (e.g. E345K/R, E430G, S440Y). In some embodiments the invention relates to Fc regions with individual alterations, such as E345K or E345R, whereas in other embodiments the invention relates to Fc regions with three alterations, such as E345R/E430G/S440Y. Additional sequence alterations (D265A and K322A) intended to decrease effector function, such a complement-dependent cytotoxicity (CDC), are shown in bold and underlined. These additional alterations may be combined with one or more hexamerization enhancing alteration. A C-terminal lysine (K) residue has been removed in FIG. 1 and SEQ ID NO: 44, as well as many of the other heavy chain and heavy chain constant region sequences disclosed in the Sequence Listing. However, in other embodiments (SEQ ID NO: 85), especially nucleic acid constructs encoding the heavy chains and heavy chain constant regions of the anti-huCD40 antibodies of the present invention, these sequences include an additional lysine residue (or codon encoding lysine) at the C-terminus of the protein (or 3′ end of the nucleotides).

FIGS. 2A and 2B show the sequences of various recombinant human immunoglobulin constant regions (residues 118-447), some of which have modified IgG2 hinge regions to increase the agonist activity of the anti-CD40 antibodies of the present invention. FIG. 2A provides the CH1 domain and hinge sequences (residues 118-327), whereas FIG. 2B provides the CH2 and CH3 domain sequences (residues 328-447). Variants of the constant regions or hybrid constant regions comprising one or more mutations are indicated on separate lines with only mutant residues indicated (in bold). Sequences with mutation(s) are omitted in regions in which there are no relevant mutations (e.g. residues 1-60 and 358-447). Other sequence variants of interest, such as C131S and C219S variants of IgG2.5 and IgG2.3, respectively, are underlined, as are all three mutations of IgG1.3f (L234A/L235E/G237A). Sequence identifiers are provided at FIG. 2B for all sequences presented in FIGS. 2A and 2B. Although the SEQ ID NOs: are only presented adjacent to residues 238-297 of each sequence, they comprise the full length sequence for that heavy chain constant region (residues 118-447). All sequences in FIGS. 2A and 2B comprise a C-terminal lysine residue that may be omitted in any final antibody, antibody formulation, or nucleic acid encoding the antibody chain, as indicated for FIG. 1.

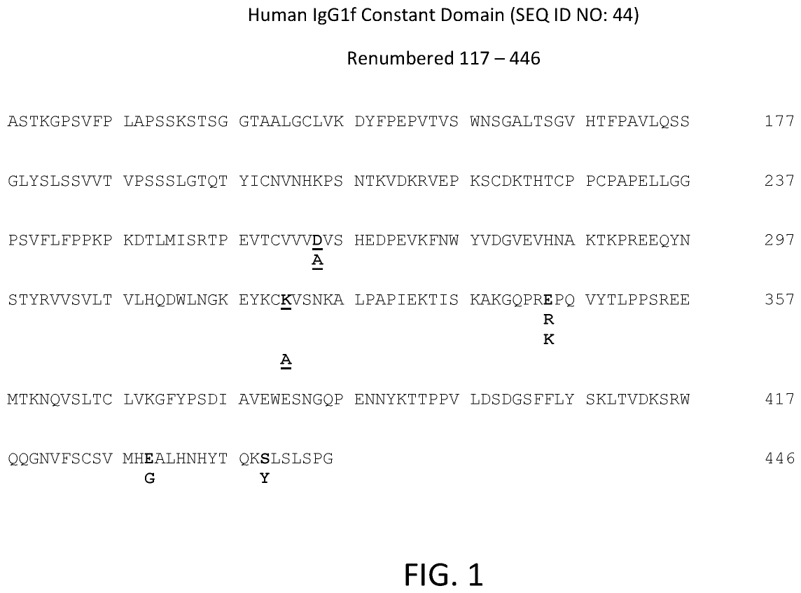

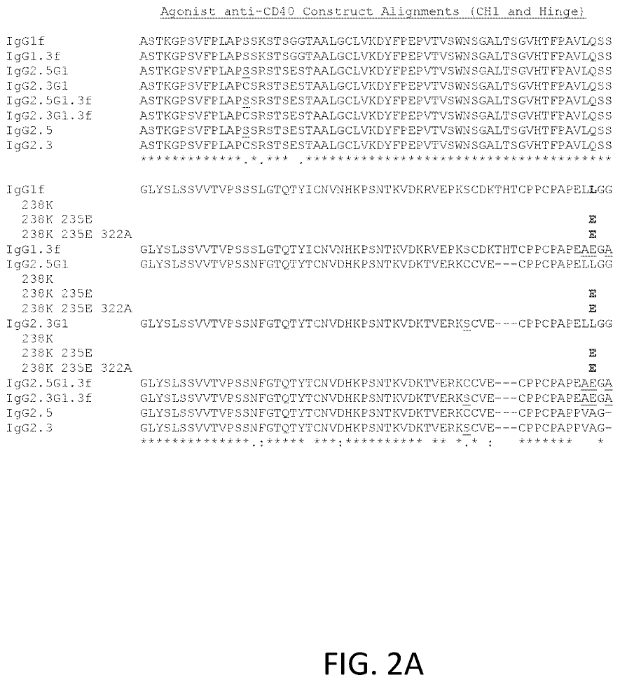

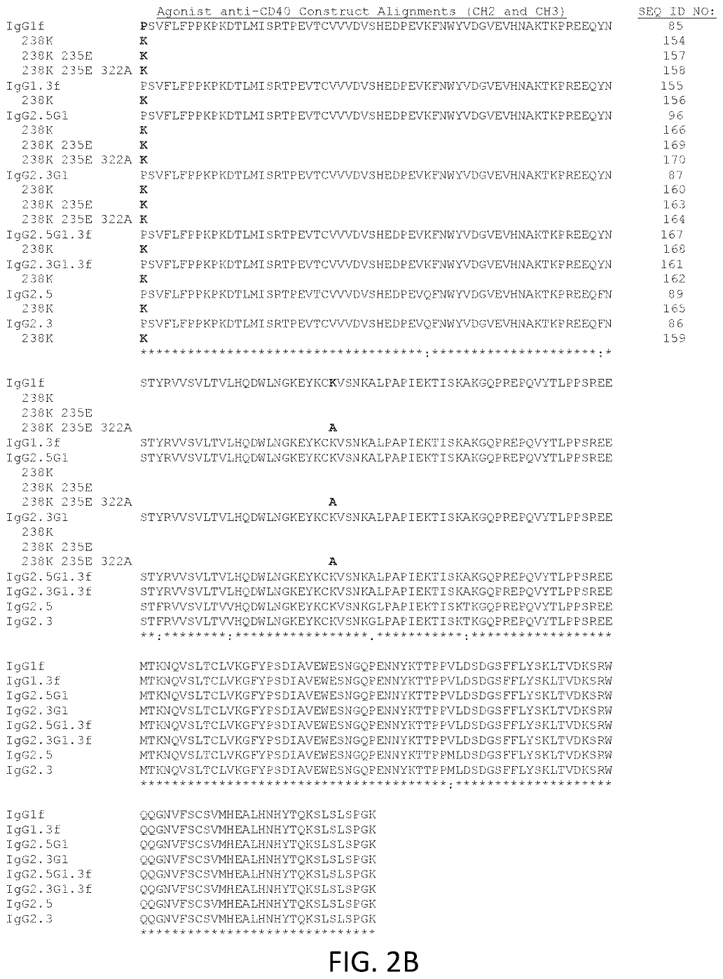

### DETAILED DESCRIPTION

The present invention provides isolated antibodies, particularly monoclonal antibodies, e.g., humanized or human monoclonal antibodies, that specifically bind to human CD40 (“huCD40”) and have enhanced agonist activity. Sequences are provided for various humanized murine anti-huCD40 monoclonal antibodies having heavy chain constant region sequence alterations that enhance their inherent agonist activity.

Further provided herein are methods of making such antibodies, immunoconjugates and bispecific molecules comprising such antibodies, and pharmaceutical compositions formulated to contain the antibodies. Also provided herein are methods of using the antibodies for immune response enhancement, alone or in combination with other immunostimulatory agents (e.g., antibodies) and/or cancer or anti-infective therapies. Accordingly, the anti-huCD40 antibodies described herein may be used in a treatment in a wide variety of therapeutic applications, including, for example, inhibiting tumor growth and treating chronic viral infections.

### Definitions

In order that the present description may be more readily understood, certain terms are first defined. Additional definitions are set forth throughout the detailed description.

CD40 refers to “TNF receptor superfamily member 5” (TNFRSF5). Unless otherwise indicated, or clear from the context, references to CD40 herein refer to human CD40 (“huCD40”), and anti-CD40 antibodies refer to anti-human CD40 antibodies. Human CD40 is further described at GENE ID NO: 958 and MIM (Mendelian Inheritance in Man): 109535. The sequence of human CD40 (NP_001241.1), including 20 amino acid signal sequence, is provided at SEQ ID NO: 1.

CD40 interacts with CD40 ligand (CD40L), which is also referred to as TNFSF5, gp39 and CD154. Unless otherwise indicated, or clear from the context, references to CD40L herein refer to human CD40L (“huCD40L”). Human CD40L is further described at GENE ID NO: 959 and MIM: 300386. The sequence of human CD40L (NP_000065.1) is provided at SEQ ID NO: 2.

An “antibody” refers, in one embodiment, to a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. In certain naturally occurring IgG, IgD and IgA antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four framework regions (FRs), arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁷ to 10⁻¹¹M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen. By way of example, an antibody that binds specifically to human CD40 might also cross-react with CD40 from certain non-human primate species (e.g., cynomolgus monkey), but might not cross-react with CD40 from other species, or with an antigen other than CD40.

Unless otherwise indicated, an immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids. Unless otherwise indicated, antibodies of the present invention comprise the IgG1f constant region (SEQ ID NO: 44) comprising sequence modifications to enhance agonist activity.

A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas. See, e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations that occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e isotype switch). Therefore, the rearranged and somatically mutated nucleic acid sequences that encode the light chain and heavy chain immunoglobulin polypeptides in response to an antigen may not be identical to the original germline sequences, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. Human antibodies of the present invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody, e.g. a mouse antibody, are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody. A “hybrid” antibody refers to an antibody having heavy and light chains of different types, such as a mouse (parental) heavy chain and a humanized light chain, or vice versa.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally occurring variants within a specific isotype group, which variants differ in one or a few amino acids. See, e.g., Jefferis et al. (2009) mAbs 1:1.

The phrases “an antibody recognizing an antigen” and “an antibody specific for an antigen” are used interchangeably herein with the term “an antibody that binds specifically to an antigen.”

An “isolated antibody,” as used herein, refers to an antibody that is substantially free of other antibodies having different antigenic specificities (e.g., an isolated antibody that specifically binds to CD40 is substantially free of antibodies that specifically bind antigens other than CD40). An isolated antibody that specifically binds to an epitope of CD40 may, however, have cross-reactivity to other CD40 proteins from different species.

“Effector functions,” deriving from the interaction of an antibody Fc region with certain Fc receptors, include but are not necessarily limited to C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with an antigen binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently FcγRIIB) receptor. Various properties of human FcγRs are summarized in Table 1. The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

TABLE 1
Properties of Human FcγRs

| | Allelic | Affinity for | | |
|---|---|---|---|---|
| Fcγ | variants | human IgG | Isotype preference | Cellular distribution |
| FcγRI | None | High | IgG1 = 3 > 4 >> 2 | Monocytes, macrophages, |
| | described | (K_{D}~10 nM) | | activated neutrophils, |
| | | | | dendritic cells? |
| FcγRIIA | H131 | Low to medium | IgG1 > 3 > 2 > 4 | Neutrophils, monocytes, |
| | | | | macrophages, eosinophils, |
| | R131 | Low | IgG1 > 3 > 4 > 2 | dendritic cells, platelets |
| FcγRIIIA | V158 | Medium | IgG1 = 3 >> 4 > 2 | NK cells, monocytes, |
| | F158 | Low | IgG1 = 3 >> 4 > 2 | macrophages, mast cells, |
| | | | | eosinophils, dendritic cells? |
| FcγRIIb | I232 | Low | IgG1 = 3 = 4 > 2 | B cells, monocytes, |
| | T232 | Low | IgG1 = 3 = 4 > 2 | macrophages, dendritic |
| | | | | cells, mast cells |

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises CH2 and CH3 constant domains in each of the antibody's two heavy chains; IgM and IgE Fc regions comprise three heavy chain constant domains (CH domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or an amino acid between these two amino acids) to the carboxy-terminus of the heavy chain, wherein the numbering is according to the EU index as in Kabat. Kabat et al. (1991) Sequences of Proteins of Immunological Interest, National Institutes of Health, Bethesda, Md.; see also FIGS. 3c-3f of U.S. Pat. App. Pub. No. 2008/0248028. As used herein, the Fc may refer to this region in isolation or in the context of an Fc-comprising protein polypeptide such as a “binding protein comprising an Fc region,” also referred to as an “Fc fusion protein” (e.g., an antibody or immunoadhesin). The boundaries of the CH1, hinge, CH2 and CH3 domains of the antibodies of the present invention are provided infra, at Table 2 and surrounding text.

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs. See, e.g., Jefferis et al. (2009) mAbs 1:1.

The term “epitope” or “antigenic determinant” refers to a site on an antigen (e.g., huCD40) to which an immunoglobulin or antibody specifically binds. Epitopes within protein antigens can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “epitope mapping” refers to the process of identification of the molecular determinants on the antigen involved in antibody-antigen recognition. Methods for determining what epitopes are bound by a given antibody are well known in the art and include, for example, immunoblotting and immunoprecipitation assays, wherein overlapping or contiguous peptides from (e.g., from CD40) are tested for reactivity with a given antibody (e.g., anti-CD40 antibody); x-ray crystallography; 2-dimensional nuclear magnetic resonance; yeast display (see example 6 of WO 2017/004006); and HDX-MS (see, e.g., Epitope Mapping Protocols in Methods in Molecular Biology, Vol. 66, G. E. Morris, Ed. (1996)) (see example 5 of WO 2017/004006).

The term “binds to the same epitope” with reference to two or more antibodies means that the antibodies bind to the same segment of amino acid residues, as determined by a given method. Techniques for determining whether antibodies bind to the “same epitope on CD40” with the antibodies described herein include, for example, epitope mapping methods, such as, x-ray analyses of crystals of antigen:antibody complexes, which provides atomic resolution of the epitope, and hydrogen/deuterium exchange mass spectrometry (HDX-MS). Other methods monitor the binding of the antibody to antigen fragments (e.g. proteolytic fragments) or to mutated variations of the antigen where loss of binding due to a modification of an amino acid residue within the antigen sequence is often considered an indication of an epitope component, such as alanine scanning mutagenesis (Cunningham & Wells (1985) Science 244:1081) or yeast display of mutant target sequence variants (see example 6 of WO 2017/004006). In addition, computational combinatorial methods for epitope mapping can also be used. These methods rely on the ability of the antibody of interest to affinity isolate specific short peptides from combinatorial phage display peptide libraries. Antibodies having the same or closely related VH and VL or the same CDR sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb. Protoc.; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al. (1983) Methods in Enzymology 9:242); solid phase direct biotin-avidin EIA (see Kirkland et al. (1986) J. Immunol. 137:3614); solid phase direct labeled assay, solid phase direct labeled sandwich assay (see Harlow and Lane (1988), Antibodies: A Laboratory Manual, Cold Spring Harbor Press); solid phase direct label RIA using I-125 label (see Morel et al. (1988) Mol. Immunol. 25(1):7); solid phase direct biotin-avidin EIA (Cheung et al. (1990) Virology 176:546); and direct labeled RIA. (Moldenhauer et al. (1990) Scand. J. Immunol. 32:77).

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., recombinant human CD40, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to human CD40” refers to an antibody that binds to soluble or cell bound human CD40 with a K_{D} of 10⁻⁷ M or less, such as approximately less than 10⁻⁸ M, 10⁻⁹ M or 10⁻¹⁰ M or even lower. An antibody that “cross-reacts with cynomolgus CD40” refers to an antibody that binds to cynomolgus CD40 with a K_{D} of 10⁻⁷ M or less, such as approximately less than 10⁻⁸ M, 10⁻⁹ M or 10⁻¹⁰ M or even lower.

The term “k_{assoc}” or “kₐ”, as used herein, refers to the association rate constant of a particular antibody-antigen interaction, whereas the term “k_{dis}” or “k_{d},” as used herein, refers to the dissociation rate constant of a particular antibody-antigen interaction. The term “K_{D}”, as used herein, refers to the equilibrium dissociation constant, which is obtained from the ratio of k_{d} to kₐ (i.e., k_{d}/kₐ) and is expressed as a molar concentration (M). K_{D} values for antibodies can be determined using methods well established in the art. A preferred method for determining the K_{D} of an antibody is biolayer interferometry (BLI) analysis, preferably using a ForteBio Octet RED device (see example 3 of WO 2017/004006), surface plasmon resonance, preferably using a biosensor system such as a BIACORE® surface plasmon resonance system (see example 4 of WO 2017/004006), or flow cytometry and Scatchard analysis.

The term “EC50” in the context of an in vitro or in vivo assay using an antibody refers to the concentration of an antibody that induces a response that is 50% of the maximal response, i.e., halfway between the maximal response and the baseline.

The term “binds to immobilized CD40” refers to the ability of an antibody described herein to bind to CD40, for example, expressed on the surface of a cell or attached to a solid support.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein may contain a modification such as, but not limited to, glycosylation, phosphorylation or a disulfide bond. A “protein” may comprise one or more polypeptides.

The term “nucleic acid molecule,” as used herein, is intended to include DNA molecules and RNA molecules. A nucleic acid molecule may be single-stranded or double-stranded, and may be cDNA.

Also provided are “conservative sequence modifications” to the antibody sequence provided herein, i.e. nucleotide and amino acid sequence modifications that do not abrogate the binding of the antibody encoded by the nucleotide sequence or containing the amino acid sequence, to the antigen. For example, modifications can be introduced by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD40 antibody is preferably replaced with another amino acid residue from the same side chain family. Methods of identifying nucleotide and amino acid conservative substitutions that do not eliminate antigen binding are well-known in the art. See, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997).

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The percent identity between two sequences is a function of the number of identical positions shared by the sequences when the sequences are optimally aligned (i.e., % homology=# of identical positions/total # of positions×100), with optimal alignment determined taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and may be a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition or depletion of a T_{reg} cell. “T effector” (“T_{eff}”) cells refers to T cells (e.g., CD4⁺ and CD8⁺ T cells) with cytolytic activities as well as T helper (Th) cells, which secrete cytokines and activate and direct other immune cells, but does not include regulatory T cells (T_{reg} cells).

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

An “immunomodulator” or “immunoregulator” refers to an agent, e.g., a component of a signaling pathway that may be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating cancer.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the terms “inhibits” or “blocks” are used interchangeably and encompass both partial and complete inhibition/blocking by at least about 50%, for example, at least about 60%, 70%, 80%, 90%, 95%, 99%, or 100%.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Prophylaxis refers to administration to a subject who does not have a disease, to prevent the disease from occurring or minimize its effects if it does.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that slows cancer progression or promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. Pharmacological effectiveness refers to the ability of the drug to promote cancer regression in the patient. Physiological safety refers to an acceptably low level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated using the assays described infra. Inhibition of tumor growth may not be immediate after treatment, and may only occur after a period of time or after repeated administration. Alternatively, this property of a composition can be evaluated by examining the ability of the compound to inhibit cell growth, such inhibition can be measured in vitro by assays known to the skilled practitioner. In other preferred embodiments described herein, tumor regression may be observed and may continue for a period of at least about 20 days, more preferably at least about 40 days, or even more preferably at least about 60 days.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g. administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

The terms “patient” and “subject” refer to any human that receives either prophylactic or therapeutic treatment. For example, the methods and compositions described herein can be used to treat a subject having cancer.

“Hinge,” “hinge domain” or “hinge region” or “antibody hinge region” refers to the domain of a heavy chain constant region that joins the CH1 domain to the CH2 domain and comprises upper, middle, and lower portions. Roux et al. (1998) J. Immunol. 161:4083. The hinge provides varying levels of flexibility, depending on sequence, between the antigen binding domain and effector region of an antibody and also provides sites for intermolecular disulfide bonding between the two heavy chain constant regions. As used herein, a hinge starts at E216 and ends at G237 for all IgG isotypes (by EU numbering). Id. The sequences of wildtype IgG1, IgG2, IgG3 and IgG4 hinges are show in Table 2.

TABLE 2
Hinge Region Sequences

| | C- | | | |
|---|---|---|---|---|
| Ig | terminal | Upper | Middle | Lower |
| Type | C_{H}1* | Hinge | Hinge | Hinge |
| IgG1 | VDKRV | EPKSCDKTHT | CPPCP | APELLGG |
| | (SEQ ID | (SEQ ID | (SEQ ID | (SEQ ID |
| | NO: 47) | NO: 48) | NO: 49) | NO: 50) |
| IgG2 | VDKTV | ERK | CCVECPPCP | APPVAG |
| | (SEQ ID | | (SEQ ID | (SEQ ID |
| | NO: 51) | | NO: 52) | NO: 53) |
| IgG3 | VDKRV | ELKTPLGDTTHT | CPRCP | APELLGG |
| (17- | (SEQ ID | (SEQ ID | (EPKSCDTP | (SEQ ID |
| 15- | NO: 47) | NO: 54) | PPCPRCP)₃ | NO: 50) |
| 15- | | | (SEQ ID | |
| 15) | | | NO: 55) | |
| IgG3 | VDKRV | ELKTPLGDTTHT | CPRCP | APELLGG |
| (17- | (SEQ ID | (SEQ ID | (EPKSCDTP | (SEQ ID |
| 15- | NO: 47) | NO: 54) | PPCPRCP)₂ | NO: 50) |
| 15) | | | (SEQ ID | |
| | | | NO: 56) | |
| IgG3 | VDKRV | ELKTPLGDTTHT | CPRCP | APELLGG |
| (17- | (SEQ ID | (SEQ ID | (EPKSCDTP | (SEQ ID |
| 15) | NO: 47) | NO: 54) | PPCPRCP)₁ | NO: 50) |
| | | | (SEQ ID | |
| | | | NO: 57) | |
| IgG3 | VDKRV | EPKS | CDTPPPCPRCP | APELLGG |
| (15- | (SEQ ID | (SEQ ID | (EPKSCDTP | (SEQ ID |
| 15- | NO: 47) | NO: 58) | PPCPRCP)₂ | NO: 50) |
| 15) | | | (SEQ ID | |
| | | | NO: 59) | |
| IgG3 | VDKRV | EPKS | CDTPPPCPRCP | APELLGG |
| (15) | (SEQ ID | (SEQ ID | (SEQ ID | (SEQ ID |
| | NO: 47) | NO: 58) | NO: 60) | NO: 50) |
| IgG4 | VDKRV | ESKYGPP | CPSCP | APEFLGG |
| | (SEQ ID | (SEQ ID | (SEQ ID | (SEQ ID |
| | NO: 47) | NO: 61) | NO: 62) | NO: 63) |

*C-terminal amino acid sequences of the CH1 domains.

The term “hinge” includes wild-type hinges (such as those set forth in Table 2), as well as variants thereof (e.g., non-naturally-occurring hinges or modified hinges). For example, the term “IgG2 hinge” includes wildtype IgG2 hinge, as shown in Table 2, and variants having 1, 2, 3, 4, 5, 1-3, 1-5, 3-5 and/or at most 5, 4, 3, 2, or 1 mutations, e.g., substitutions, deletions or additions. Exemplary IgG2 hinge variants include IgG2 hinges in which 1, 2, 3 or all 4 cysteines (C219, C220, C226 and C229) are changed to another amino acid, e.g. serine. In a specific embodiment, the IgG2 hinge region comprises a C219S substitution. In certain embodiments, the hinge comprises sequences from at least two isotypes. For example, the hinge may comprise the upper, middle or lower hinge from one isotype and the remainder of the hinge from one or more other isotypes. For example, the hinge can be an IgG2/IgG1 hinge, and may comprise, e.g., the upper and middle hinges of IgG2 and the lower hinge of IgG1. A hinge may have effector function or be deprived of effector function. For example, the lower hinge of wildtype IgG1 provides effector function. The term “CH1 domain” refers to the heavy chain constant region linking the variable domain to the hinge in a heavy chain constant domain. As used herein, a CH1 domain starts at A118 and ends at V215. The term “CH2 domain” refers to the heavy chain constant region linking the hinge to the CH3 domain in a heavy chain constant region. As used herein, a CH2 domain starts at P238 and ends at K340. The term “CH3 domain” refers to the heavy chain constant region that is C-terminal to the CH2 domain in a heavy chain constant region. As used herein, a CH3 domain starts at G341 and ends at K447.

Various aspects described herein are described in further detail in the following subsections.

### I. Anti-CD40 Antibodies

The present application discloses agonistic anti-huCD40 antibodies having desirable properties for use as therapeutic agents in treating diseases such as cancers. These properties include one or more of the ability to bind to human CD40 with high affinity, acceptably low immunogenicity in human subjects, and the absence of sequence liabilities that might reduce the chemical stability of the antibody. These antibodies further comprise one or more mutations in the constant region that enhance the ability of the antibodies to aggregate, e.g. into hexamers, which enhances the agonist activity beyond the agonist activity inherent in the parent antibody lacking the mutations in the constant region. The antibodies may optionally further comprise one or more mutations in the constant region that decrease effector function, such as ADCC or CDC. In some embodiments, agonist anti-CD40 antibodies of the present invention aggregate when bound to CD40 on a cell surface and are “silent” or “inert” with regard to effector function. Any reduction or elimination of effector function is measured by reference to an otherwise identical antibody having a wildtype human IgG1 constant region, such as human IgG1f (SEQ ID NO: 44 or 85).

An ADCC reporter bioassay may be used to determine whether an antibody exhibits reduced ADCC. In such an assay a test antibody is exposed to CD40-expressing cell, and then to an engineered effector cell line expressing i) an NFAT response element upstream of luciferase, and ii) FcγRIIIa receptor. Fc receptor binding, a surrogate for ADCC activity, is measured by detecting luminescence such that an antibody with reduced ADCC activity produces a lower luminescence signal. An antibody of the present invention is considered to have reduced ADCC activity if it is at least 2-fold less active than an otherwise identical antibody having a wildtype human IgG1f constant region, in an ADCC reporter assay of the type described. CDC activity may be measured by detecting binding to complement protein c1q, e.g. by surface plasmon resonance (SPR) or by ELISA. An antibody exhibiting a 2-fold reduction in affinity for c1q compared an otherwise identical antibody having a wildtype human IgG1f constant region in such an assay would be considered to exhibit reduced CDC.

Anti-huCD40 Antibodies that Compete with Anti-huCD40 Antibodies Disclosed Herein

Anti-huCD40 antibodies that compete with the antibodies of the present invention for binding to huCD40 may be raised using immunization protocols similar to those described at examples 1 and 2 of WO 2017/004006. Antibodies that compete for binding with the anti-huCD40 antibodies disclosed herein by sequence may also be generated by immunizing mice or other non-human animal with human CD40 or a construct comprising the extracellular domain thereof (residues 21-193 of SEQ ID NO: 1), or by immunizing with a fragment of human CD40 containing the epitope bound by the anti-huCD40 antibodies disclosed herein. The resulting antibodies can be screened for the ability to block binding of 12D6, 5F11, 8E8, 5G7 and/or 19G3 to human CD40 by methods well known in the art, for example blocking binding to fusion protein of the extracellular domain of CD40 and an immunoglobulin Fc domain in a ELISA, or blocking the ability to bind to cells expressing huCD40 on their surface, e.g. by FACS. In various embodiments, the test antibody is contacted with the CD40-Fc fusion protein (or to cells expressing huCD40 on their surface) prior to, at the same time as, or after the addition of 12D6, 5F11, 8E8, 5G7 or 19G3. For example, “binning” experiments may be performed to determine whether a test antibody falls into the same “bin” as an antibodies disclosed herein by sequence, with antibodies disclosed herein by sequence as the “reference” antibodies and the antibodies to be tested as the “test” antibodies. Antibodies that reduce binding of the antibodies disclosed herein by sequence to human CD40 (either as an Fc fusion or on a cell), particularly at roughly stoichiometric concentrations, are likely to bind at the same, overlapping, or adjacent epitopes, and thus may share the desirable functional properties of 12D6, 5F11, 8E8, 5G7 or 19G3.

Accordingly, provided herein are anti-huCD40 antibodies that inhibit the binding of an anti-huCD40 antibodies described herein to huCD40 on cells by at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, or by 100%, and/or whose binding to huCD40 on cells is inhibited by an anti-huCD40 antibodies described herein by at least 10%, 20%, 30%, 40%, 50%, 55%, 60%, 65%, 70%, 75%, 80%, 85%, 90%, 91%, 92%, 93%, 94%, 95%, 96%, 97%, 98%, 99%, or by 100%, e.g., as measured by ELISA or FACS, such as by using the assay described in the following paragraph.

An exemplary competition experiment to determine whether a test antibody blocks the binding of (i.e., “competes with”) a reference antibody, may be conducted as follows: cells expressing CD40 are seeded at 10⁵ cells per sample well in a 96 well plate. The plate is set on ice followed by the addition of unconjugated test antibody at concentrations ranging from 0 to 50 μg/mL (three-fold titration starting from a highest concentration of 50 μg/mL). An unrelated IgG may be used as an isotype control for the first antibody and added at the same concentrations (three-fold titration starting from a highest concentration of 50 μg/mL). A sample pre-incubated with 50 μg/mL unlabeled reference antibody may be included as a positive control for complete blocking (100% inhibition) and a sample without antibody in the primary incubation may be used as a negative control (no competition; 0% inhibition). After 30 minutes of incubation, labeled, e.g., biotinylated, reference antibody is added at a concentration of 2 μg/mL per well without washing. Samples are incubated for another 30 minutes on ice. Unbound antibodies are removed by washing the cells with FACS buffer. Cell-bound labeled reference antibody is detected with an agent that detects the label, e.g., PE conjugated streptavidin (Invitrogen, catalog #S21388) for detecting biotin. The samples are acquired on a FACS Calibur Flow Cytometer (BD, San Jose) and analyzed with FlowJo software (Becton, Dickinson & Company, Ashland, Oreg.). The results may be represented as the % inhibition (i.e., subtracting from 100% the amount of label at each concentration divided by the amount of label obtained with no blocking antibody).

Typically, the same experiment is then conducted in the reverse, i.e., the test antibody is the reference antibody and the reference antibody is the test antibody. In certain embodiments, an antibody at least partially (e.g., at least 10%, 20%, 30%, 40%, 50%, 60%, 70%, 80%, or 90%) or completely (100%) blocks the binding of the other antibody to the target, e.g. human CD40, and regardless of whether inhibition occurs when one or the other antibody is the reference antibody. A reference antibody and a test antibody “cross-block” binding of each other to the target when the antibodies compete with each other both ways, i.e., in competition experiments in which the reference antibody is added first and in competition experiments in which the test antibody is added first.

Anti-huCD40 antibodies are considered to compete with the anti-huCD40 antibodies disclosed herein if they inhibit binding of 12D6, 5F11, 8E8, 5G7 and/or 19G3 to human CD40 by at least 10%, 20%, 30%, 40%, 50%, 60%, 70%, 80%, 90%, or by 100%, when present at roughly equal concentrations, for example in competition experiments like those described in example 4 of WO 2017/004006. Unless otherwise indicated, an antibody will be considered to compete with an antibody selected from the group consisting of the anti-CD40 antibodies of the present invention if it reduces binding of the selected antibody to human CD40 (SEQ ID NO: 1) by at least 20% when used at a roughly equal molar concentration with the selected antibody, as measured in competition ELISA experiments as outlined in the preceding two paragraphs.

Anti-huCD40 Antibodies that Bind to the Same Epitope

Anti-huCD40 antibodies that bind to the same or similar epitopes to the antibodies disclosed herein may be raised using standard immunization protocols. The resulting antibodies can be screened for high affinity binding to human CD40. Selected antibodies can then be studied in yeast display assay in which sequence variants of huCD40 are presented on the surface of yeast cells, or by hydrogen-deuterium exchange experiments, to determine the precise epitope bound by the antibody. See, e.g., WO 2017/004006.

Epitope determinations may be made by any method known in the art. In various embodiments, anti-huCD40 antibodies are considered to bind to the same epitope as an anti-huCD40 mAb disclosed herein if they make contact with one or more of the same residues within at least one region of huCD40; if they make contacts with a majority of the residues within at least one region of huCD40; if they make contacts with a majority of the residues within each region of huCD40; if they make contact with a majority of contacts along the entire length of huCD40; if they make contacts within all of the same distinct regions of human CD40; if they make contact with all of the residues at any one region on human CD40; or if they make contact with all of the same residues at all of the same regions. Epitope “regions” are clusters of residues along the primary sequence.

Techniques for determining antibodies that bind to the “same epitope on huCD40” with the antibodies described herein include x-ray analyses of crystals of antigen:antibody complexes, which provides atomic resolution of the epitope. Other methods monitor the binding of the antibody to antigen fragments or mutated variations of the antigen where loss of binding due to a modification of an amino acid residue within the antigen sequence is often considered an indication of an epitope component. Methods may also rely on the ability of an antibody of interest to affinity isolate specific short peptides (either in native three dimensional form or in denatured form) from combinatorial phage display peptide libraries or from a protease digest of the target protein. The peptides are then regarded as leads for the definition of the epitope corresponding to the antibody used to screen the peptide library. For epitope mapping, computational algorithms have also been developed that have been shown to map conformational discontinuous epitopes.

The epitope or region comprising the epitope can also be identified by screening for binding to a series of overlapping peptides spanning CD40. Alternatively, the method of Jespers et al. (1994) Biotechnology 12:899 may be used to guide the selection of antibodies having the same epitope and therefore similar properties to the an anti-CD40 antibodies described herein. Using phage display, first the heavy chain of the anti-CD40 antibody is paired with a repertoire of (preferably human) light chains to select a CD40-binding antibody, and then the new light chain is paired with a repertoire of (preferably human) heavy chains to select a (preferably human) CD40-binding antibody having the same epitope or epitope region as an anti-huCD40 antibody described herein. Alternatively variants of an antibody described herein can be obtained by mutagenesis of cDNA encoding the heavy and light chains of the antibody.

Alanine scanning mutagenesis, as described by Cunningham & Wells (1989) Science 244: 1081, or some other form of point mutagenesis of amino acid residues in CD40 (such as the yeast display method provided at example 6 of WO 2017/004006) may also be used to determine the functional epitope for an anti-CD40 antibody.

The epitope or epitope region (an “epitope region” is a region comprising the epitope or overlapping with the epitope) bound by a specific antibody may also be determined by assessing binding of the antibody to peptides comprising fragments of CD40. A series of overlapping peptides encompassing the sequence of CD40 (e.g., human CD40) may be synthesized and screened for binding, e.g. in a direct ELISA, a competitive ELISA (where the peptide is assessed for its ability to prevent binding of an antibody to CD40 bound to a well of a microtiter plate), or on a chip. Such peptide screening methods may not be capable of detecting some discontinuous functional epitopes, i.e. functional epitopes that involve amino acid residues that are not contiguous along the primary sequence of the CD40 polypeptide chain.

An epitope may also be identified by MS-based protein footprinting, such as hydrogen/deuterium exchange mass spectrometry (HDX-MS) and Fast Photochemical Oxidation of Proteins (FPOP). HDX-MS may be conducted, e.g., as further described at Wei et al. (2014) Drug Discovery Today 19:95, the methods of which are specifically incorporated by reference herein. See also example 5 of WO 2017/004006. FPOP may be conducted as described, e.g., in Hambley & Gross (2005) J American Soc. Mass Spectrometry 16:2057, the methods of which are specifically incorporated by reference herein.

The epitope bound by anti-CD40 antibodies may also be determined by structural methods, such as X-ray crystal structure determination (e.g., WO2005/044853), molecular modeling and nuclear magnetic resonance (NMR) spectroscopy, including NMR determination of the H-D exchange rates of labile amide hydrogens in CD40 when free and when bound in a complex with an antibody of interest (Zinn-Justin et al. (1992) Biochemistry 31:11335; Zinn-Justin et al. (1993) Biochemistry 32:6884).

With regard to X-ray crystallography, crystallization may be accomplished using any of the known methods in the art (e.g. Giege et al. (1994) Acta Crystallogr. D 50:339; McPherson (1990) Eur. J. Biochem. 189:1), including microbatch (e.g. Chayen (1997) Structure 5:1269), hanging-drop vapor diffusion (e.g. McPherson (1976) J. Biol. Chem. 251:6300), seeding and dialysis. It is desirable to use a protein preparation having a concentration of at least about 1 mg/mL and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent, e.g. glycerol at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 3.0 to about 5.0, preferably about 4.0. Specific buffers useful in the precipitant solution may vary and are well-known in the art (Scopes, Protein Purification: Principles and Practice, Third ed., (1994) Springer-Verlag, New York). Examples of useful buffers include, but are not limited to, HEPES, Tris, IVIES and acetate. Crystals may be grow at a wide range of temperatures, including 2° C., 4° C., 8° C. and 26° C.

Antibody:antigen crystals may be studied using well-known X-ray diffraction techniques and may be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al., eds., Academic Press; U.S. Patent Application Publication No. 2004/0014194), and BUSTER (Bricogne (1993) Acta Cryst. D 49:37-60; Bricogne (1997) Meth. Enzymol. 276A:361-423, Carter & Sweet, eds.; Roversi et al. (2000) Acta Cryst. D 56:1313-1323), the disclosures of which are hereby incorporated by reference in their entireties. Unless otherwise indicated, and with reference to the claims, the epitope bound by an antibody is the epitope as determined by HDX-MS methods, substantially as described in example 5 of WO 2017/004006.

Anti-CD40 Antibodies that Bind with High Affinity

In some embodiments the anti-huCD40 antibodies of the present invention bind to huCD40 with high affinity, like the anti-huCD40 antibodies disclosed herein, increasing their likelihood of being effective therapeutic agents. In various embodiments anti-huCD40 antibodies of the present invention bind to huCD40 with a K_{D} of less than 10 nM, 5 nM, 2 nM, 1 nM, 300 pM or 100 pM. In other embodiments, the anti-huCD40 antibodies of the present invention bind to huCD40 with a K_{D} between 2 nM and 100 pM. Standard assays to evaluate the binding ability of the antibodies toward huCD40 include ELISAs, RIAs, Western blots, biolayer interferometry (BLI) and BIACORE® SPR analysis. See, e.g., WO 2017/004006.

### Anti-CD40 Antibody Sequence Variants

Some variability in the antibody sequences disclosed herein may be tolerated and still maintain the desirable properties of the antibody. The CDR regions are delineated using the Kabat system (Kabat, E. A., et al. (1991) Sequences of Proteins of Immunological Interest, Fifth Edition, U.S. Department of Health and Human Services, NIH Publication No. 91-3242). Accordingly, the present invention further provides anti-huCD40 antibodies comprising CDR sequences that are at least 70%, 75%, 80%, 85%, 90%, 95%, 96%, 97%, 98%, or 99% identical to the CDR sequences of the antibodies disclosed herein (i.e. 12D6, 5F11, 8E8, 5G7 and 19G3 and humanized derivatives thereof). The present invention also provides anti-huCD40 antibodies comprising heavy and/or light chain variable domain sequences that are at least 70%, 75%, 80%, 85%, 90%, 95%, 96%, 97%, 98%, or 99% identical to the heavy and/or light chain variable domain sequences of the antibodies disclosed herein (i.e. 12D6, 5F11, 8E8, 5G7 and 19G3 and humanized derivatives thereof).

Anti-CD40 Antibodies Sharing CDR Sequences or Derived from the Same Murine Germlines

Given that antigen-binding specificity is determined primarily by the CDRs, antibodies sharing CDRs sequences with antibodies disclosed herein (i.e. 12D6, 5F11, 8E8, 5G7 and 19G3) are likely to share their desirable properties. In some embodiments, anti-huCD40 antibodies of the present invention comprises heavy and light chain variable regions derived from the same murine V region and J region germline sequences as antibody 12D6, 5F11, 8E8, 5G7 or 19G3. Antibody 12D6 has a heavy chain derived from murine germlines VH1-39_01 and IGHJ4, and light chain germlines VK1-110_01 and IGKJ1. Antibody 5F11 has a heavy chain derived from murine germlines VH1-4_02 and IGHJ3, and light chain germlines VK3-5_01 and IGKJ5. Antibody 8E8 has a heavy chain derived from murine germlines VH1-80_01 and IGHJ2, and light chain germlines VK1-110_01 and IGKJ2. Antibody 5G7 has a heavy chain derived from murine germlines VH1-18_01 and IGHJ4, and light chain germlines VK10-96_01 and IGKJ2. Antibody 19G3 has a heavy chain derived from murine germlines VH5-9-4_01 and IGHJ3, and light chain germlines VK1-117_01 and IGKJ2. Heavy chain D region germline sequences (making up part of CDRH3) are not specified, as they are often difficult to assign given their high variability, and thus antibodies of the present invention may comprise heavy chains derived from the listed V and J region germlines and any D region germline. Other antibodies that bind to human CD40 and are derived from some or all of these germline sequences are likely to be closely related in sequence, particularly those derived from the same V-region genes, and thus would be expected to share the same desirable properties.

As used herein, a murine antibody comprises heavy or light chain variable regions that are “derived from” a particular germline sequence if the variable regions of the antibody are obtained from a system that uses murine germline immunoglobulin genes, and the antibody sequence is sufficiently related to the germline that it is more likely derived from the given germline than from any other. Such systems include immunizing a mouse with the antigen of interest. The murine germline immunoglobulin sequence(s) from which the sequence of an antibody is “derived” can be identified by comparing the amino acid sequence of the antibody to the amino acid sequences of murine germline immunoglobulins and selecting the germline immunoglobulin sequence that is closest in sequence (i.e., greatest % identity) to the sequence of the antibody. A murine antibody that is “derived from” a particular germline immunoglobulin sequence may contain amino acid differences as compared to the germline sequence due to, for example, naturally-occurring somatic mutations or intentional introduction of site-directed mutation. However, a selected murine antibody typically is at least 90% identical in amino acids sequence to an amino acid sequence encoded by a germline immunoglobulin gene (e.g. V regions). In certain cases, a murine antibody may be at least 95%, or even at least 96%, 97%, 98%, or 99% identical in amino acid sequence to the amino acid sequence encoded by the germline immunoglobulin gene (e.g. V regions). Typically, an antibody derived from a particular murine germline sequence will display no more than 10 amino acid differences from the amino acid sequence encoded by the germline immunoglobulin gene (e.g. V regions). In certain cases, the murine antibody may comprise no more than 5, or even no more than 4, 3, 2, or 1 amino acid difference from the amino acid sequence encoded by the germline immunoglobulin gene (e.g. V regions).

### II. Engineered and Modified Antibodies to Enhance Agonist Activity

### Fc Regions Modified for Hexamerization

Antibodies of the present invention may comprise the variable domains of the invention combined with constant regions comprising Fc regions with amino acid substitutions that enhance hexamerization of the antibodies. Such mutations in include E345K, E345R, E430G and S440Y. Studies have found that such mutations, alone or in combination, enhance hexamer formation in antibodies, e.g. human IgG1 antibodies. Diebolder et al. (2014) Science 343:1260; de Jong et al. (2016) PLoS Biol. 14(1):e1002344 (publ. 6 Jan. 2016); Zhang et al. (2016) JBC 291:27134; WO 14/06217; WO 14/108198; WO 2016/164480. T437R and K248E mutations have been shown to have similar effects. Zhang et al. (2017) mAbs DOI 10.1080/19420862.2017.1358838). Others had demonstrated that cross-linked anti-CD40 antibodies exhibit enhanced FcγR-independent agonist activity, suggesting that anti-CD40 antibodies with intrinsic cross-linking ability might provide high levels of immunostimulation. White et al. (2014) J Immunol. 193:1828. Such findings are consistent with a model in which receptor multimerization (clustering) on the cell surface is required for activation of immunostimulatory TNF-family receptors. White et al. (2014) J. Immunol. 193:1828. Such FcγR-independent agonist activity may be particularly advantageous in treating tumors with low levels of FcγR-expressing cells in the tumor microenvironment. White et al. (2014) J. Immunol. 193:1828; Zhang et al. (2017) mAbs DOI 10.1080/19420862.2017.1358838).

In various embodiments of the present invention, humanized agonist anti-CD40 antibodies are modified to incorporate one or more of the mutations E345K, E345R, E430G and S440Y, such as E345K alone, E345R alone or E345R/E430G/S440Y. Single mutations may be advantageous for therapeutic use because they promote aggregation primarily once antibody is bound to CD40 on a cell surface, whereas the triple mutant E345R/E430G/S440Y promotes hexamerization in solution, and thus potential undesirable aggregation, even in the absence of bound antigen. Diebolder et al. (2014) Science 343:1260; WO 14/06217.

Some of these and other constructs of the present invention are provided at Table 3. Full sequence identifiers are provided for some of the constructs, and others provide a sequence identifier number with a plus sign (e.g. 44+), which indicates that the complete variant sequence can be constructed by adding the mutations listed in the table to the identified sequence from the sequence listing. In all sequences of the present invention, even if not expressly indicated, E345R may be replaced with E345K and vice versa.

TABLE 3
Additional Hexamer Constant Region Sequence Variants

| | SEQ | | | | | | |
|---|---|---|---|---|---|---|---|
| Designation | ID: | P238 | D265 | K322 | E345 | E430 | S440 |
| 12D6-hIgG1 E345R | 172 | | | | R | | |
| 12D6-hIgG1.3 E345R | 175 | | | | R | | |
| 12D6-hIgG1.3 E345R K322A | 177 | | | A | R | | |
| 12D6-hIgG1 P238K E345R | 44+ | K | | | R | | |
| 12D6-hIgG1 P238K E345R K322A | 44+ | K | | A | R | | |
| 12D6-hIgG1 E345K | 171 | | | | K | | |
| 12D6-hIgG1.3 E345K | 174 | | | | K | | |
| 12D6-hIgG1.3 E345K K322A | 155+ | | | A | K | | |
| 12D6-hIgG1 P238K E345K | 44+ | K | | | K | | |
| 12D6-hIgG1 P238K E345K K322A | 44+ | K | | A | K | | |
| 12D6-hIgG1 E430G | 44+ | | | | | G | |
| 12D6-hIgG1.3 E430G | 155+ | | | | | G | |
| 12D6-hIgG1.3 E430G K322A | 155+ | | | A | | G | |
| 12D6-hIgG1 P238K E430G | 44+ | K | | | | G | |
| 12D6-hIgG1 P238K E430G K322A | 44+ | K | | A | | G | |
| 12D6-hIgG1 E345R E430G S440Y | 173 | | | | R | G | Y |
| 12D6-hIgG1.3 E345R E430G S440Y | 176 | | | | R | G | Y |
| 12D6-hIgG1.3 E345R E430G S440Y K322A | 155+ | | | A | R | G | Y |
| 12D6-hIgG1 P238K E345R E430G S440Y | 44+ | K | | | R | G | Y |
| 12D6-hIgG1 P238K E345R E430G S440Y K322A | 44+ | K | | A | R | G | Y |
| 12D6-hIgG2 E345R | 186+ | | | | R | | |
| 12D6-hIgG2 D265A E345R | 186+ | | A | | R | | |
| 12D6-hIgG2.3 D265A E345R | 86+ | | A | | R | | |
| 12D6-hIgG2.5 D265A E345R | 89+ | | A | | R | | |
| 12D6-hIgG2 P238K E345R | 186+ | | | | R | | |
| 12D6-hG2.3 P238K E345R | 86+ | K | | | R | | |
| 12D6-hG2.5 P238K E345R | 89+ | K | | | R | | |
| 12D6-hIgG2 E345R K322A | 186+ | | | A | R | | |
| 12D6-hIgG2 D265A E345R K322A | 186+ | | A | A | R | | |
| 12D6-hIgG2.3 D265A E345R K322A | 86+ | | A | A | R | | |
| 12D6-hIgG2.5 D265A E345R K322A | 89+ | | A | A | R | | |
| 12D6-hIgG2 P238K E345R K322A | 186+ | K | | A | R | | |
| 12D6-hG2.3 P238K E345R K322A | 86+ | K | | A | R | | |
| 12D6-hG2.5 P238K E345R K322A | 89+ | K | | A | R | | |
| 12D6-hIgG2 E345K | 186+ | | | | K | | |
| 12D6-hIgG2 D265A E345K | 186+ | | A | | K | | |
| 12D6-hIgG2.3 D265A E345K | 86+ | | A | | K | | |
| 12D6-hIgG2.5 D265A E345K | 89+ | | A | | K | | |
| 12D6-hIgG2 P238K E345K | 186+ | K | | | K | | |
| 12D6-hG2.3 P238K E345K | 86+ | K | | | K | | |
| 12D6-hG2.5 P238K E345K | 89+ | K | | | K | | |
| 12D6-hIgG2 E345K K322A | 186+ | | | A | K | | |
| 12D6-hIgG2 D265A E345K K322A | 186+ | | A | A | K | | |
| 12D6-hIgG2.3 D265A E345K K322A | 86+ | | A | A | K | | |
| 12D6-hIgG2.5 D265A E345K K322A | 89+ | | A | A | K | | |
| 12D6-hIgG2 P238K E345K K322A | 186+ | K | | A | K | | |
| 12D6-hG2.3 P238K E345K K322A | 86+ | K | | A | K | | |
| 12D6-hG2.5 P238K E345K K322A | 89+ | K | | A | K | | |
| 12D6-hIgG2 E430G | 186+ | | | | | G | |
| 12D6-hIgG2 D265A E430G | 186+ | | A | | | G | |
| 12D6-hIgG2.3 D265A E430G | 86+ | | A | | | G | |
| 12D6-hIgG2.5 D265A E430G | 89+ | | A | | | G | |
| 12D6-hIgG2 P238K E430G | 186+ | K | | | | G | |
| 12D6-hG2.3 P238K E430G | 86+ | K | | | | G | |
| 12D6-hG2.5 P238K E430G | 89+ | K | | | | G | |
| 12D6-hIgG2 E430G K322A | 186+ | | | A | | G | |
| 12D6-hIgG2 D265A E430G K322A | 186+ | | A | A | | G | |
| 12D6-hIgG2.3 D265A E430G K322A | 86+ | | A | A | | G | |
| 12D6-hIgG2.5 D265A E430G K322A | 89+ | | A | A | | G | |
| 12D6-hIgG2 P238K E430G K322A | 186+ | K | | A | | G | |
| 12D6-hG2.3 P238K E430G K322A | 86+ | K | | A | | G | |
| 12D6-hG2.5 P238K E430G K322A | 89+ | K | | A | | G | |
| 12D6-hIgG2 E345R E430G S440Y | 186+ | | | | R | G | Y |
| 12D6-hIgG2 D265A E345R E430G S440Y | 186+ | | A | | R | G | Y |
| 12D6-hIgG2.3 D265A E345R E430G S440Y | 86+ | | A | | R | G | Y |
| 12D6-hIgG2.5 D265A E345R E430G S440Y | 89+ | | A | | R | G | Y |
| 12D6-hIgG2 P238K E345R E430G S440Y | 186+ | K | | | R | G | Y |
| 12D6-hG2.3 P238K E345R E430G S440Y | 86+ | K | | | R | G | Y |
| 12D6-hG2.5 P238K E345R E430G S440Y | 89+ | K | | | R | G | Y |
| 12D6-hIgG2 E345R E430G S440Y K322A | 186+ | | | A | R | G | Y |
| 12D6-hIgG2 D265A E345R E430G S440Y K322A | 186+ | | A | A | R | G | Y |
| 12D6-hIgG2.3 D265A E345R E430G S440Y K322A | 86+ | | A | A | R | G | Y |
| 12D6-hIgG2.5 D265A E345R E430G S440Y K322A | 89+ | | A | A | R | G | Y |

Non-IgG2 Heavy Chain Constant Regions with IgG2 Hinge Regions

In various embodiments, anti-CD40 antibodies of the present invention include constant region sequence modifications that enhance antibody aggregation, or stabilize inherently more agonistic antibody conformations, thus providing FcγR-independent enhancement of agonism. Exemplary anti-CD40 antibodies with modified IgG2 domains providing such enhanced agonism are described at WO 2015/145360 and White et al. (2015) Cancer Cell 27:138, the disclosures of which are hereby incorporated by reference in their entireties. Anti-CD40 antibody CP870,893 is an IgG2 antibody having FcγR-independent agonistic activity. White et al. (2015) Cancer Cell 27:138. Such FcγR-independent agonism may be advantageous in treating some tumors in which there are few FcγR-expressing cells (e.g. few NK cells or macrophages), although it may also lead to undesirable CD40 agonism outside the tumor microenvironment. White et al. (2014) J. Immunol. 193:1828; Zhang et al. (2017) mAbs DOI 10.1080/19420862.2017.1358838).

The present invention provides anti-CD40 antibodies comprising the CDR or variable domain sequences disclosed herein linked to non-hIgG2 heavy chain constant regions (e.g. IgG1) having hIgG2 hinge regions, or variants thereof, including CH1 domain sequence variants. Such “IgG2 hinge” antibodies exhibit enhanced agonism compared with antibodies with a fully IgG1 heavy chain constant region, which enhanced agonism is independent of Fcγ receptor-mediated cross-linking. In preferred embodiments, these IgG2 hinge anti-CD40 antibodies retain substantially unchanged antigen binding affinity. The present invention provides methods of enhancing FcγR-independent agonism of non-IgG2 anti-CD40 antibodies comprising replacing the non-IgG2 hinge with an IgG2 hinge. In certain embodiments, a modified heavy chain constant region comprises a hinge of the IgG2 isotype (an “IgG2 hinge”) and a CH1, CH2 and CH3 domain, wherein at least one of the CH1, CH2 and CH3 domains is not of the IgG2 isotype. The IgG2 hinge may be a wildtype human IgG2 hinge (e.g., ERKCCVECPPCPAPPVAG; SEQ ID NO: 77) or a variant thereof that also confers enhanced agonist activity. In certain embodiments, such IgG2 hinge variants have similar rigidity or stiffness as wildtype IgG2 hinge. The rigidity of a hinge can be determined, e.g., by computer modeling, electron microscopy, spectroscopy such as Nuclear Magnetic Resonance (NMR), X-ray crystallography (B-factors), or Sedimentation Velocity Analytical ultracentrifugation (AUC) to measure or compare the radius of gyration of antibodies comprising the hinge. Exemplary human IgG2 hinge variants comprise substitution(s) of one or more of the four cysteine residues (i.e., C219, C220, C226 and C229), for example with serine. One exemplary IgG2 hinge variant comprises a C219S mutation (e.g., ERKSCVECPPCPAPPVAG; SEQ ID NO: 78). Other IgG2 hinge variants comprise C220S, C226S or C229S mutation, any of which may be combined with a C219S mutation.

An IgG2 hinge variant may also comprise non-IgG2 hinge sequence elements (a “chimeric hinge”), provided that the rigidity of the chimeric hinge is at least similar to that of a wildtype IgG2 hinge. For example, in one embodiment, an IgG2 hinge variant comprises a wildtype IgG1 lower hinge. See Table 2.

Table 4 provides exemplary “IgG2 hinge” human heavy chain constant region sequences differing in the isotypic origins of the CH1, CH2 and CH3 domains. As used herein, “IgG2 hinge antibody” refers not just to antibodies comprising hinge regions derived from IgG2, but also CH1 regions derived from IgG2 (SEQ ID NO: 186). An unfilled cell in Table 4 indicates that the indicated domain may be of any isotype, or may be completely absent. In certain embodiments, a modified heavy chain constant region comprises a variant CH1 domain, e.g. including A114C and/or T173C mutations. A modified heavy chain constant region may also comprise a variant CH2 domain, e.g. including A330S and/or P331S mutations.

TABLE 4
Exemplary “IgG2 Hinge” Human Heavy
Chain Constant Region Constructs

| CH1 | Hinge | CH2 | CH3 |
|---|---|---|---|
| | IgG2 | | |
| IgG1 | IgG2 | | |
| IgG2 | IgG2 | | |
| | IgG2 | IgG1 | |
| | IgG2 | IgG2 | |
| | IgG2 | | IgG1 |
| | IgG2 | | IgG2 |
| IgG1 | IgG2 | IgG1 | |
| IgG1 | IgG2 | IgG2 | |
| IgG2 | IgG2 | IgG1 | |
| IgG2 | IgG2 | IgG2 | |
| IgG1 | IgG2 | | IgG1 |
| IgG1 | IgG2 | | IgG2 |
| IgG2 | IgG2 | | IgG1 |
| IgG2 | IgG2 | | IgG2 |
| | IgG2 | IgG1 | IgG1 |
| | IgG2 | IgG1 | IgG2 |
| | IgG2 | IgG2 | IgG1 |
| | IgG2 | IgG2 | IgG2 |
| IgG1 | IgG2 | IgG1 | IgG1 |
| IgG1 | IgG2 | IgG1 | IgG2 |
| IgG1 | IgG2 | IgG2 | IgG1 |
| IgG1 | IgG2 | IgG2 | IgG2 |
| IgG2 | IgG2 | IgG1 | IgG1 |
| IgG2 | IgG2 | IgG1 | IgG2 |
| IgG2 | IgG2 | IgG2 | IgG1 |
| IgG2 | IgG2 | IgG2 | IgG2 |

Selected specific exemplary antibody constant regions comprising combinations of IgG2 CH1 and hinge sequences with other isotype sequences, and select amino acid substitutions, are provided at Table 5.

TABLE 5
Selected Exemplary “IgG2 Hinge”
Human Heavy Chain Constant Regions

| Construct | SEQ ID NO: | Description |
|---|---|---|
| IgG1f | 85 | wild type IgG1f |
| IgG1.1f | 90 | standard inert IgG1.1f |
| IgG2.3 | 86 | IgG2 A-form (C219S) |
| IgG2.5 | 89 | IgG2 B-form (C131S) |
| IgG2.3G1-KH | 88 | CH1, upper hinge and lower |
| | | hinge/upper CH2 of IgG2.3, all |
| | | else IgG1f |
| IgG2.5G1-KH | 97 | CH1, upper hinge and lower |
| | | hinge/upper CH2 of IgG2.5, all |
| | | else IgG1f |
| IgG2.3G1-AY | 87 | CH1 and upper hinge of IgG2.3, |
| | | all else IgG1f |
| IgG2.5G1-AY | 96 | CH1 and upper hinge of IgG2.5, |
| | | all else IgG1f |
| IgG1-G2.3G1-KH | 100 | CH1 of IgG1, upper hinge and |
| | | lower hinge/upper CH2 of |
| | | IgG2.3, all else IgG1f |
| IgG1-G2.3G1-AY | 99 | CH1 of IgG1, upper hinge of IgG2.3, |
| | | all else IgG1f |
| IgG2.3G1.1f-KH | 91 | CH1, upper hinge and lower |
| | | hinge/upper CH2 of IgG2.3, all |
| | | else IgG1.1f |
| IgG2.5G1.1f-KH | 95 | CH1, upper hinge and lower |
| | | hinge/upper CH2 of IgG2.5, all |
| | | else IgG1.1f |
| IgG1-deltaTHT | 91 | IgG1 with THT sequence removed |
| | | from hinge |
| IgG2.3-plusTHT | 93 | IgG2.3 with THT sequence (from IgG1) |
| | | added into hinge |
| IgG2.5-plusTHT | 98 | IgG2.5 with THT sequence (from IgG1) |
| | | added into hinge |
| IgG2.3-plusGGG | 94 | IgG2.3 with flexible GGG sequence |
| | | added into hinge |

Additional specific exemplary antibody constant regions comprising combinations of IgG2 CH1 and hinge sequences with other isotype sequences, and select amino acid substitutions, are provided at Table 6.

TABLE 6
Additional Exemplary “IgG2 Hinge”
Human Heavy Chain Constant Regions

| Construct | SEQ ID NO: | Description |
|---|---|---|
| G2-G1-G1-G1 | 101 | CH1 domain of IgG2, with all |
| G2.5-G1-G1-G1 | 102 | else IgG1. Also, Cys > Ser |
| | | mutant to reduce potential |
| | | disulfide heterogeneity. |
| G1-G2.3-G2-G2 | 103 | CH1 domain of IgG1 with all |
| | | else IgG2.3 |
| G1-KRGEGSSNLF | 104 | Swap CH1 regions in IgG1 with |
| G1-KRGEGS | 105 | those of IgG2, either |
| G1-SNLF | 106 | separately or together. |
| IgG1-ITNDRTPR | 107 | |
| G1-SNLFPR | 108 | |
| G2-RKEGSGNSFL | 109 | Swap CH1 regions in IgG2 with |
| G2-RKEGSG | 110 | those of IgG1, either |
| G2-NSFL | 111 | separately or together. |
| IgG2-TIDNTRRP | 112 | |
| G2-NSFLRP | 113 | |
| G1-G1-G2-G1-AY | 114 | IgG1 with CH2 domain residues |
| G1-G1-G2-G1-KH | 115 | of IgG2 |
| G2-G2.3-G1-G2-KH | 116 | IgG2 with CH2 domain residues |
| G2.5-G2.3-G1-G2-KH | 117 | of IgG1 |
| G2-G2.3-G1-G2-AY | 118 | |
| G2.5-G2.3-G1-G2-AY | 119 | |
| G1-G2.3-G1-G1-KH | 120 | Swap hinge regions between |
| G2-G1-G2-G2-AY | 121 | IgG1 and IgG2. |
| G2.5-G1-G2-G2-AY | 122 | |
| G1-G2-G1-G1-AY | 123 | |
| G2-G1-G2-G2-KH | 124 | |
| G2.5-G1-G2-G2-KH | 125 | |
| IgG1-deltaHinge | 126 | Hinge truncations |
| IgG2-deltaHinge | 127 | |
| IgG2.5-deltaHinge | 128 | |
| IgG1-deltaG237 | 129 | |
| IgG2-plusG237 | 130 | |
| IgG2.4 | 131 | Other |
| IgG2.3/4 | 132 | |

Anti-CD40 antibodies of the present invention, including antibodies comprising the CDR and/or variable domain sequences disclosed herein, may incorporate the “IgG2 hinge” constant region sequences disclosed herein, e.g. to enhance FcgR-independent agonist activity. Such IgG2 hinge constant regions include those disclosed at Table 5 (SEQ ID NOs: 86-89 and 91-100) and Table 6 (SEQ ID NOs: 101-132), and also those disclosed at SEQ ID NOs: 69-76.

Additional IgG2 hinge constructs are provided at SEQ ID NOs: 159-170, and more specifically at SEQ ID NOs: 159-161 and 165-167. Such constructs comprise hybrid IgG sequences with IgG2 CH1 and hinge regions, combined with mutations (P238K or the triple mutation L234A/L235E/G237A of IgG1.3f) to reduce or eliminate unwanted effector function. These constructs attempt to provide a pure CD40 agonist activity without causing effector functions that would deplete the CD40⁺ cells bound by the antibody.

### Targeted Antigen Binding

In various embodiments, the antibody of the present invention is modified to selectively block antigen binding in tissues and environments where antigen binding would be detrimental, but allow antigen binding where it would be beneficial. In one embodiment, a blocking peptide “mask” is generated that specifically binds to the antigen binding surface of the antibody and interferes with antigen binding, which mask is linked to each of the binding arms of the antibody by a peptidase cleavable linker. See, e.g., U.S. Pat. No. 8,518,404 to CytomX. Such constructs are useful for treatment of cancers in which protease levels are greatly increased in the tumor microenvironment compared with non-tumor tissues. Selective cleavage of the cleavable linker in the tumor microenvironment allows disassociation of the masking/blocking peptide, enabling antigen binding selectively in the tumor, rather than in peripheral tissues in which antigen binding might cause unwanted side effects.

Alternatively, in a related embodiment, a bivalent binding compound (“masking ligand”) comprising two antigen binding domains is developed that binds to both antigen binding surfaces of the (bivalent) antibody and interfere with antigen binding, in which the two binding domains masks are linked to each other (but not the antibody) by a cleavable linker, for example cleavable by a peptidase. See, e.g., Intl Pat. App. Pub. No. WO 2010/077643 to Tegopharm Corp. Masking ligands may comprise, or be derived from, the antigen to which the antibody is intended to bind, or may be independently generated. Such masking ligands are useful for treatment of cancers in which protease levels are greatly increased in the tumor microenvironment compared with non-tumor tissues. Selective cleavage of the cleavable linker in the tumor microenvironment allows disassociation of the two binding domains from each other, reducing the avidity for the antigen-binding surfaces of the antibody. The resulting dissociation of the masking ligand from the antibody enables antigen binding selectively in the tumor, rather than in peripheral tissues in which antigen binding might cause unwanted side effects.

### Fc Variants to Reduce or Eliminate Effector Function

In various embodiments of the present invention, the antibodies are further modified to reduce or eliminate unwanted effector functions, such as ADCC or CDC. Such modifications include amino acid substitutions (also referred to herein as mutations) and alterations in glycosylation. The anti-CD40 antibodies of the present invention are agonists, and thus are intended to elicit CD40 signaling in the CD40-expressing cells to which they bind. Depletion of these CD40-expressing cells would be counterproductive since it would eliminate the very cells whose biological activity is being relied on to help mount an anti-tumor (or anti-viral infected cell) immune response.

In some embodiments of the present invention are further modified to reduce unwanted effector function. In some such embodiments the lower hinge region includes the three mutations L234A, L235E and G237A, referred to as IgG1.3f (SEQ ID NO: 155). In other such embodiments, the antibodies comprise the P238K mutation, optionally further comprising the L235E mutation, and further optionally including the K322A mutation (to reduce complement binding). D265A may also be introduced to reduce effector function. Such mutations will reduce or eliminate various effector functions, while leaving the pure agonist activity of the anti-CD40 antibodies.

### Additional Fc Variants

When using an IgG4 constant region, it is usually preferable to include the substitution S228P, which mimics the hinge sequence in IgG1 and thereby stabilizes IgG4 molecules, e.g. reducing Fab-arm exchange between the therapeutic antibody and endogenous IgG4 in the patient being treated. Labrijn et al. (2009) Nat. Biotechnol. 27:767; Reddy et al. (2000) J. Immunol. 164:1925. In some embodiments the IgG2 portion of the constant region is modified to replace a cysteine residue with a serine residue to minimize disulfide shuffling, which can lead to undesired heterogeneity in an antibody preparation. In one embodiment the IgG2 CH1 domain comprises a C131S mutation (IgG2.5; SEQ ID NO: 89), and in another embodiment the IgG2 upper hinge region comprises a C219S mutation (IgG2.3; SEQ ID NO: 86).

A potential protease cleavage site in the hinge of IgG1 constructs can be eliminated by D221G and K222S modifications, increasing the stability of the antibody. WO 2014/043344.

The affinities and binding properties of an Fc variant for its ligands (Fc receptors) may be determined by a variety of in vitro assay methods (biochemical or immunological based assays) known in the art including but not limited to, equilibrium methods (e.g., enzyme-finked immunosorbent assay (ELISA), or radioimmunoassay (RIA)), or kinetics (e.g., BIACORE® SPR analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions.

### III. Nucleic Acid Molecules

Another aspect described herein pertains to nucleic acid molecules that encode the antibodies described herein. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and may or may not contain intronic sequences.

### IV. C-Terminal Lysine

The N- and C-termini of antibody polypeptide chains of the present invention may differ from the expected sequence due to commonly observed post-translational modifications. For example, C-terminal lysine residues are often missing from antibody heavy chains. Dick et al. (2008) Biotechnol. Bioeng. 100:1132. N-terminal glutamine residues, and to a lesser extent glutamate residues, are frequently converted to pyroglutamate residues on both light and heavy chains of therapeutic antibodies. Dick et al. (2007) Biotechnol. Bioeng. 97:544; Liu et al. (2011) JBC 28611211; Liu et al. (2011) J. Biol. Chem. 286:11211.

Amino acid sequences for various agonist anti-huCD40 antibodies of the present invention are provided in the Sequence Listing, which is summarized at Table 9. For the reasons mentioned above, the C-terminal lysine is not included in many of sequences in the Sequence Listing for heavy chains or heavy chain constant regions. However, in an alternative embodiment, each heavy chain for the anti-huCD40 antibodies of the present invention, and/or genetic construct encoding such antibodies or the heavy or light chains thereof, includes this additional lysine residue at the C-terminus of the heavy chain(s).

### V. Assays

Antibodies described herein can be tested for binding to CD40 by, for example, standard ELISA. Briefly, microtiter plates are coated with purified CD40 at 1-2 μg/ml in PBS, and then blocked with 5% bovine serum albumin in PBS. Dilutions of antibody (e.g., dilutions of plasma from CD40-immunized mice) are added to each well and incubated for 1-2 hours at 37° C. The plates are washed with PBS/Tween and then incubated with secondary reagent (e.g., for human antibodies, or antibodies otherwise having a human heavy chain constant region, a goat-anti-human IgG Fc-specific polyclonal reagent) conjugated to horseradish peroxidase (HRP) for 1 hour at 37° C. After washing, the plates are developed with ABTS substrate (Moss Inc, product: ABTS-1000) and analyzed by a spectrophotometer at OD 415-495. Sera from immunized mice are then further screened by flow cytometry for binding to a cell line expressing human CD40, but not to a control cell line that does not express CD40. Briefly, the binding of anti-CD40 antibodies is assessed by incubating CD40 expressing CHO cells with the anti-CD40 antibody at 1:20 dilution. The cells are washed and binding is detected with a PE-labeled anti-human IgG Ab. Flow cytometric analyses are performed using a FACScan flow cytometry (Becton Dickinson, San Jose, Calif.). Preferably, mice that develop the highest titers will be used for fusions. Analogous experiments may be performed using anti-mouse detection antibodies if mouse anti-huCD40 antibodies are to be detected.

An ELISA as described above can be used to screen for antibodies and, thus, hybridomas that produce antibodies that show positive reactivity with the CD40 immunogen. Hybridomas that produce antibodies that bind, preferably with high affinity, to CD40 can then be subcloned and further characterized. One clone from each hybridoma, which retains the reactivity of the parent cells (by ELISA), can then be chosen for making a cell bank, and for antibody purification.

To purify anti-CD40 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected anti-CD40 monoclonal antibodies bind to unique epitopes, each antibody can be biotinylated using commercially available reagents (Pierce, Rockford, Ill.). Biotinylated MAb binding can be detected with a streptavidin labeled probe. Competition studies using unlabeled monoclonal antibodies and biotinylated monoclonal antibodies can be performed using CD40 coated-ELISA plates as described above.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

To test the binding of monoclonal antibodies to live cells expressing CD40, flow cytometry can be used. Briefly, cell lines expressing membrane-bound CD40 (grown under standard growth conditions) are mixed with various concentrations of monoclonal antibodies in PBS containing 0.1% BSA at 4° C. for 1 hour. After washing, the cells are reacted with Phycoerythrin (PE)-labeled anti-IgG antibody under the same conditions as the primary antibody staining. The samples can be analyzed by FACScan instrument using light and side scatter properties to gate on single cells and binding of the labeled antibodies is determined. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Anti-huCD40 antibodies can be further tested for reactivity with the CD40 antigen by Western blotting. Briefly, cell extracts from cells expressing CD40 can be prepared and subjected to sodium dodecyl sulfate polyacrylamide gel electrophoresis. After electrophoresis, the separated antigens will be transferred to nitrocellulose membranes, blocked with 20% mouse serum, and probed with the monoclonal antibodies to be tested. IgG binding can be detected using anti-IgG alkaline phosphatase and developed with BCIP/NBT substrate tablets (Sigma Chem. Co., St. Louis, Mo.).

Methods for analyzing binding affinity, cross-reactivity, and binding kinetics of various anti-CD40 antibodies include standard assays known in the art, for example, Biolayer Interferometry (BLI) analysis, and BIACORE® surface plasmon resonance (SPR) analysis using a BIACORE® 2000 SPR instrument (Biacore AB, Uppsala, Sweden).

In one embodiment, an antibody specifically binds to the extracellular region of human CD40. An antibody may specifically bind to a particular domain (e.g., a functional domain) within the extracellular domain of CD40. In certain embodiments, the antibody specifically binds to the extracellular region of human CD40 and the extracellular region of cynomolgus CD40. Preferably, an antibody binds to human CD40 with high affinity.

### VI. Bispecific Molecules

Antibodies described herein may be used for forming bispecific molecules. An anti-CD40 antibody, can be derivatized or linked to another functional molecule, e.g., another peptide or protein (e.g., another antibody or ligand for a receptor) to generate a bispecific molecule that binds to at least two different binding sites or target molecules. The antibody described herein may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, peptide or binding mimetic, such that a bispecific molecule results.

Accordingly, provided herein are bispecific molecules comprising at least one first binding specificity for CD40 and a second binding specificity for a second target epitope. In an embodiment described herein in which the bispecific molecule is multispecific, the molecule can further include a third binding specificity.

While human monoclonal antibodies are preferred, other antibodies that can be employed in the bispecific molecules described herein are murine, chimeric and humanized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### VII. Compositions

Further provided are compositions, e.g., a pharmaceutical compositions, containing one or more anti-CD40 antibodies, as described herein, formulated together with a pharmaceutically acceptable carrier. Such compositions may include one or a combination of (e.g., two or more different) antibodies, or immunoconjugates or bispecific molecules described herein. For example, a pharmaceutical composition described herein can comprise a combination of antibodies (or immunoconjugates or bispecifics) that bind to different epitopes on the target antigen or that have complementary activities.

In certain embodiments, a composition comprises an anti-CD40 antibody at a concentration of at least 1 mg/ml, 5 mg/ml, 10 mg/ml, 50 mg/ml, 100 mg/ml, 150 mg/ml, 200 mg/ml, or at 1-300 mg/ml or 100-300 mg/ml.

Pharmaceutical compositions described herein also can be administered in combination therapy, i.e., combined with other agents. For example, the combination therapy can include an anti-CD40 antibody described herein combined with at least one other anti-cancer and/or T-cell stimulating (e.g., activating) agent. Examples of therapeutic agents that can be used in combination therapy are described in greater detail below in the section on uses of the antibodies described herein.

In some embodiments, therapeutic compositions disclosed herein can include other compounds, drugs, and/or agents used for the treatment of cancer. Such compounds, drugs, and/or agents can include, for example, chemotherapy drugs, small molecule drugs or antibodies that stimulate the immune response to a given cancer. In some instances, therapeutic compositions can include, for example, one or more of an anti-CTLA-4 antibody, an anti-PD-1 antibody, an anti-PD-L1 antibody, an anti-TIGIT antibody, an anti-OX40 (also known as CD134, TNFRSF4, ACT35 and/or TXGP1L) antibody, an anti-LAG-3 antibody, an anti-CD73 antibody, an anti-CD137 antibody, an anti-CD27 antibody, an anti-CSF-1R antibody, a TLR agonist, or a small molecule antagonist of IDO or TGFβ.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzyl ethylenediamine, N-methylglucamine, chloroprocaine, choline, diethanolamine, ethylenediamine, procaine and the like.

A pharmaceutical composition described herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient that can be combined with a carrier material to produce a single dosage form will vary depending upon the subject being treated, and the particular mode of administration. The amount of active ingredient that can be combined with a carrier material to produce a single dosage form will generally be that amount of the composition that produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months.

In some methods, two or more monoclonal antibodies with different binding specificities are administered simultaneously, in which case the dosage of each antibody administered falls within the ranges indicated. A therapeutic antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can optionally be administered a prophylactic regime, although in many immune-oncology indications continued treatment is not necessary.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-CD40 antibody described herein preferably results in a decrease in severity of disease symptoms, an increase in frequency and duration of disease symptom-free periods, or a prevention of impairment or disability due to the disease affliction. In the context of cancer, a therapeutically effective dose preferably prevents further deterioration of physical symptoms associated with cancer. Symptoms of cancer are well-known in the art and include, for example, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like. Therapeutic efficacy may be observable immediately after the first administration of an agonistic anti-huCD40 mAb of the present invention, or it may only be observed after a period of time and/or a series of doses. Such delayed efficacy my only be observed after several months of treatment, up to 6, 9 or 12 months. It is critical not to decide prematurely that an agonistic anti-huCD40 mAb of the present invention lacks therapeutically efficacy in light of the delayed efficacy exhibited by some immune-oncology agents.

A therapeutically effective dose may prevent or delay onset of cancer, such as may be desired when early or preliminary signs of the disease are present. Laboratory tests utilized in the diagnosis of cancer include chemistries (including the measurement of soluble CD40 or CD40L levels)(Hock et al. (2006) Cancer 106:2148; Chung & Lim (2014) J. Trans. Med. 12:102), hematology, serology and radiology. Accordingly, any clinical or biochemical assay that monitors any of the foregoing may be used to determine whether a particular treatment is a therapeutically effective dose for treating cancer. One of ordinary skill in the art would be able to determine such amounts based on such factors as the subject's size, the severity of the subject's symptoms, and the particular composition or route of administration selected.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with anti-huCD40 antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-huCD40 antibodies described herein can be formulated to ensure proper distribution in vivo. For example, the blood-brain barrier (BBB) excludes many highly hydrophilic compounds. To ensure that the therapeutic compounds described herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### VIII. Uses and Methods

The antibodies, antibody compositions and methods described herein have numerous in vitro and in vivo utilities involving, for example, enhancement of immune response by agonizing CD40 signaling. In a preferred embodiment, the antibodies described herein are human or humanized antibodies. For example, anti-huCD40 antibodies described herein can be administered to cells in culture, in vitro or ex vivo, or to human subjects, e.g., in vivo, to enhance immunity in a variety of diseases. Accordingly, provided herein are methods of modifying an immune response in a subject comprising administering to the subject an antibody, described herein such that the immune response in the subject is enhanced, stimulated or up-regulated.

Preferred subjects include human patients in whom enhancement of an immune response would be desirable. The methods are particularly suitable for treating human patients having a disorder that can be treated by augmenting an immune response (e.g., the T-cell mediated immune response). In a particular embodiment, the methods are particularly suitable for treatment of cancer in vivo. To achieve antigen-specific enhancement of immunity, anti-huCD40 antibodies described herein can be administered together with an antigen of interest or the antigen may already be present in the subject to be treated (e.g., a tumor-bearing or virus-bearing subject). When antibodies to CD40 are administered together with another agent, the two can be administered separately or simultaneously.

Also encompassed are methods for detecting the presence of human CD40 antigen in a sample, or measuring the amount of human CD40 antigen, comprising contacting the sample, and a control sample, with a human monoclonal antibody, that specifically binds to human CD40, under conditions that allow for formation of a complex between the antibody and human CD40. The formation of a complex is then detected, wherein a difference complex formation between the sample compared to the control sample is indicative the presence of human CD40 antigen in the sample. Moreover, the anti-CD40 antibodies described herein can be used to purify human CD40 via immunoaffinity purification.

Given the ability of anti-huCD40 antibodies described herein to enhance co-stimulation of T cell responses, e.g., antigen-specific T cell responses, provided herein are in vitro and in vivo methods of using the antibodies described herein to stimulate, enhance or upregulate antigen-specific T cell responses, e.g., anti-tumor T cell responses.

CD4⁺ and CD8⁺ T cell responses can be enhanced using anti-CD40 antibodies. The T cells can be T_{eff} cells, e.g., CD4+ T_{eff} cells, CD8+ T_{eff} cells, T helper (Tₕ) cells and T cytotoxic (T_{c}) cells.

Further encompassed are methods of enhancing an immune response (e.g., an antigen-specific T cell response) in a subject comprising administering an anti-huCD40 antibody described herein to the subject such that an immune response (e.g., an antigen-specific T cell response) in the subject is enhanced. In a preferred embodiment, the subject is a tumor-bearing subject and an immune response against the tumor is enhanced. A tumor may be a solid tumor or a liquid tumor, e.g., a hematological malignancy. In certain embodiments, a tumor is an immunogenic tumor. In certain embodiments, a tumor is non-immunogenic. In certain embodiments, a tumor is PD-L1 positive. In certain embodiments a tumor is PD-L1 negative. A subject may also be a virus-bearing subject and an immune response against the virus is enhanced.

Further provided are methods for inhibiting growth of tumor cells in a subject comprising administering to the subject an anti-huCD40 antibody described herein such that growth of the tumor is inhibited in the subject. Also provided are methods of treating chronic viral infection in a subject comprising administering to the subject an anti-huCD40 antibody described herein such that the chronic viral infection is treated in the subject.

In certain embodiments, an anti-huCD40 antibody is given to a subject as an adjunctive therapy. Treatments of subjects having cancer with an anti-huCD40 antibody may lead to a long-term durable response relative to the current standard of care; long term survival of at least 1, 2, 3, 4, 5, 10 or more years, recurrence free survival of at least 1, 2, 3, 4, 5, or 10 or more years. In certain embodiments, treatment of a subject having cancer with an anti-huCD40 antibody prevents recurrence of cancer or delays recurrence of cancer by, e.g., 1, 2, 3, 4, 5, or 10 or more years. An anti-CD40 treatment can be used as a primary or secondary line of treatment.

These and other methods described herein are discussed in further detail below.

### Cancer

Provided herein are methods for treating a subject having cancer, comprising administering to the subject an anti-huCD40 antibody described herein, such that the subject is treated, e.g., such that growth of cancerous tumors is inhibited or reduced and/or that the tumors regress. An anti-huCD40 antibody can be used alone to inhibit the growth of cancerous tumors. Alternatively, an anti-huCD40 antibody can be used in conjunction with another agent, e.g., other immunogenic agents, standard cancer treatments, or other antibodies, as described below. Combination with an inhibitor of PD-1, such as an anti-PD-1 or anti-PD-L1 antibody, is also provided. See. e.g., Ellmark et al. (2015) Oncolmmunology 4:7 e1011484.

Accordingly, provided herein are methods of treating cancer, e.g., by inhibiting growth of tumor cells, in a subject, comprising administering to the subject a therapeutically effective amount of an anti-huCD40 antibody described herein, e.g., a humanized form of 12D6, 5F11, 8E8, 5G7 or 19G3, further comprising Fc region sequence modification(s) of the present invention to enhance agonist activity. The antibody may be a humanized anti-huCD40 antibody (such as any of the humanized anti-huCD40 antibodies described herein), a human chimeric anti-huCD40 antibody, or a humanized non-human anti-huCD40 antibody, e.g., a human, chimeric or humanized anti-huCD40 antibody that competes for binding with, or binds to the same epitope as, at least one of the anti-huCD40 antibodies specifically described herein.

Cancers whose growth may be inhibited using the antibodies of the invention include cancers typically responsive to immunotherapy. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (M0), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

Notwithstanding the above, the agonist anti-huCD40 antibodies of the present invention will not find use in treating hematologic cancers with CD40 expression, which might be exacerbated by treatment with a CD40 agonist. Certain cancers may be known to express CD40 and thus be subject to such exacerbation, and thus may be categorically excluded. In other embodiments specific tumor samples are tested for expression of CD40 and are excluded from therapy with the agonist anti-huCD40 antibodies of the present invention based on the test results.

An anti-huCD40 antibody can be administered as a monotherapy, or as the only immunostimulating therapy, or it can be combined with an immunogenic agent in a cancer vaccine strategy, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination. Dranoff et al. (1993) Proc. Natl. Acad. Sci. U.S.A. 90: 3539-43.

The study of gene expression and large scale gene expression patterns in various tumors has led to the definition of so called tumor specific antigens. Rosenberg, S A (1999) Immunity 10: 281-7. In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. CD40 agonists can be used in conjunction with a collection of recombinant proteins and/or peptides expressed in a tumor in order to generate an immune response to these proteins. These proteins are normally viewed by the immune system as self antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen that can be used in conjunction with CD40 inhibition is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with CD40 agonism to activate (unleash) more potent anti-tumor responses.

Agonism of CD40 can also be combined with standard cancer treatments (e.g., surgery, radiation, and chemotherapy). Agonism of CD40 can be effectively combined with chemotherapeutic regimes. In these instances, it may be possible to reduce the dose of chemotherapeutic reagent administered (Mokyr et al. (1998) Cancer Research 58: 5301-5304). An example of such a combination is an anti-huCD40 antibody in combination with decarbazine for the treatment of melanoma. Another example of such a combination is an anti-huCD40 antibody in combination with interleukin-2 (IL-2) for the treatment of melanoma. The scientific rationale behind the combined use of CD40 agonists and chemotherapy is that cell death, that is a consequence of the cytotoxic action of most chemotherapeutic compounds, should result in increased levels of tumor antigen in the antigen presentation pathway. Other combination therapies that may result in synergy with CD40 agonism through cell death are radiation, surgery, and hormone deprivation. Each of these protocols creates a source of tumor antigen in the host. Angiogenesis inhibitors can also be combined with CD40 agonists. Inhibition of angiogenesis leads to tumor cell death which may feed tumor antigen into host antigen presentation pathways.

The anti-huCD40 antibodies described herein can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. The T cell arm of these responses would be augmented by agonism of CD40. Alternatively, antigen may be delivered directly to DCs by the use of bispecific antibodies that bind to tumor antigen and a dendritic cell specific cell surface marker.

Tumors evade host immune surveillance by a large variety of mechanisms. Many of these mechanisms may be overcome by the inactivation of immunosuppressive proteins expressed by the tumors. These include among others TGF-β (Kehrl et al. (1986) J Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with anti-huCD40 antibodies to counteract the effects of the immunosuppressive agent and favor tumor immune responses by the host.

Anti-CD40 antibodies are able to substitute effectively for T cell helper activity. Ridge et al. (1998) Nature 393: 474-478. Activating antibodies to T cell costimulatory molecules such as CTLA-4 (e.g., U.S. Pat. No. 5,811,097), OX-40 (Weinberg et al. (2000) Immunol 164: 2160-2169), CD137/4-1BB (Melero et al. (1997) Nature Medicine 3: 682-685 (1997), and ICOS (Hutloff et al. (1999) Nature 397: 262-266) may also provide for increased levels of T cell activation. Inhibitors of PD1 or PD-L1 may also be used in conjunction with anti-huCD40 antibodies.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285: 546-51). These methods can also be used to activate T cell responses to infectious agents such as CMV. Ex vivo activation in the presence of anti-CD40 antibodies can increase the frequency and activity of the adoptively transferred T cells.

### Chronic Viral Infections

In another aspect, the invention described herein provides a method of treating an infectious disease in a subject comprising administering to the subject an anti-huCD40 antibody such that the subject is treated for the infectious disease.

Similar to its application to tumors as discussed above, antibody-mediated CD40 agonism can be used alone, or as an adjuvant, in combination with vaccines, to enhance the immune response to pathogens, toxins, and self-antigens. Examples of pathogens for which this therapeutic approach can be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. CD40 agonism is particularly useful against established infections by agents such as HIV that present altered antigens over the course of the infections. These novel epitopes are recognized as foreign at the time of anti-human CD40 antibody administration, thus provoking a strong T cell response.

Some examples of pathogenic viruses causing infections treatable by methods described herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods described herein include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lyme disease bacteria.

Some examples of pathogenic fungi causing infections treatable by methods described herein include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections treatable by methods described herein include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis.

In all of the above methods, CD40 agonism can be combined with other forms of immunotherapy such as cytokine treatment (e.g., interferons, GM-CSF, G-CSF, IL-2), or bispecific antibody therapy, which provides for enhanced presentation of tumor antigens. See, e.g., Holliger (1993) Proc. Natl. Acad. Sci. USA 90:6444-6448; Poljak (1994) Structure 2:1121-1123.

### Vaccine Adjuvants

Anti-huCD40 antibodies described herein can be used to enhance antigen-specific immune responses by co-administration of an anti-huCD40 antibody with an antigen of interest, e.g., a vaccine. Accordingly, provided herein are methods of enhancing an immune response to an antigen in a subject, comprising administering to the subject: (i) the antigen; and (ii) an anti-huCD40 antibody such that an immune response to the antigen in the subject is enhanced. The antigen can be, for example, a tumor antigen, a viral antigen, a bacterial antigen or an antigen from a pathogen. Non-limiting examples of such antigens include those discussed in the sections above, such as the tumor antigens (or tumor vaccines) discussed above, or antigens from the viruses, bacteria or other pathogens described above.

Suitable routes of administering the antibody compositions (e.g., human monoclonal antibodies, multispecific and bispecific molecules and immunoconjugates) described herein in vivo and in vitro are well known in the art and can be selected by those of ordinary skill. For example, the antibody compositions can be administered by injection (e.g., intravenous or subcutaneous). Suitable dosages of the molecules used will depend on the age and weight of the subject and the concentration and/or formulation of the antibody composition.

As previously described, anti-huCD40 antibodies described herein can be co-administered with one or other more therapeutic agents, e.g., a cytotoxic agent, a radiotoxic agent or an immunosuppressive agent. The antibody can be linked to the agent (as an immuno-complex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, dacarbazine and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days. Co-administration of anti-CD40 antibodies described herein with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells that would render them unreactive with the antibody.

Also within the scope described herein are kits comprising the antibody compositions described herein (e.g., human antibodies, bispecific or multispecific molecules, or immunoconjugates) and instructions for use. The kit can further contain at least one additional reagent, or one or more additional human antibodies described herein (e.g., a human antibody having a complementary activity that binds to an epitope in CD40 antigen distinct from the first human antibody). Kits typically include a label indicating the intended use of the contents of the kit. The term label includes any writing, or recorded material supplied on or with the kit, or that otherwise accompanies the kit.

### Combination Therapies

In addition to the combinations therapies provided above, anti-CD40 antibodies described herein can also be used in additional methods of combination therapy, e.g., for treating cancer, as described below. The present invention provides methods of combination therapy in which an anti-huCD40 antibody is co-administered with one or more additional agents, e.g., antibodies, that are effective in stimulating immune responses to thereby further enhance, stimulate or upregulate immune responses in a subject.

Generally, an anti-huCD40 antibody described herein can be combined with (i) an agonist of another co-stimulatory receptor and/or (ii) an antagonist of an inhibitory signal on T cells, either of which results in amplifying antigen-specific T cell responses (immune checkpoint regulators). Most of the co-stimulatory and co-inhibitory molecules are members of the immunoglobulin super family (IgSF), and anti-CD40 antibodies described herein may be administered with an agent that targets a member of the IgSF family to increase an immune response. One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which include CD40 and CD40L, OX-40, OX-40L, CD70, CD27L, CD30, CD30L, 4-1BBL, CD137/4-1BB, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 1β2, FAS, FASL, RELT, DR6, TROY, NGFR (see, e.g., Tansey (2009) Drug Discovery Today 00:1).

In another aspect, anti-huCD40 antibodies can be used in combination with antagonists of cytokines that inhibit T cell activation (e.g., IL-6, IL-10, TGF-ß, VEGF; or other “immunosuppressive cytokines,” or cytokines that stimulate T cell activation, for stimulating an immune response, e.g., for treating proliferative diseases, such as cancer.

In one aspect, T cell responses can be stimulated by a combination of the anti-huCD40 mAbs of the present invention and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Exemplary agents that modulate one of the above proteins and may be combined with agonist anti-huCD40 antibodies, e.g., those described herein, for treating cancer, include: YERVOY®/ipilimumab or tremelimumab (to CTLA-4), galiximab (to B7.1), BMS-936558 (to PD-1), pidilizumab/CT-011 (to PD-1), KEYTRUDA®/pembrolizumab/MK-3475 (to PD-1), AMP224 (to B7-DC/PD-L2), BMS-936559 (to B7-H1), MPDL3280A (to B7-H1), MEDI-570 (to ICOS), AMG557 (to B7H2), MGA271 (to B7H3-WO 11/109400), IMP321 (to LAG-3), urelumab/BMS-663513 and PF-05082566 (to CD137/4-1BB), varlilumab/CDX-1127 (to CD27), MEDI-6383 and MEDI-6469 (to OX40), RG-7888 (to OX40L-WO 06/029879), Atacicept (to TACI), muromonab-CD3 (to CD3).

Other molecules that can be combined with agonist anti-huCD40 antibodies for the treatment of cancer include antagonists of inhibitory receptors on NK cells or agonists of activating receptors on NK cells. For example, agonist anti-huCD40 antibodies can be combined with antagonists of KIR (e.g., lirilumab).

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

Generally, agonist anti-huCD40 antibodies described herein can be used together with one or more of agonistic agents that ligate positive co-stimulatory receptors, blocking agents that attenuate signaling through inhibitory receptors, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit T_{regs} (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

Provided herein are methods for stimulating an immune response in a subject comprising administering to the subject a CD40 agonist, e.g., an antibody, and one or more additional immunostimulatory antibodies, such as a PD-1 antagonist, e.g., antagonist antibody, a PD-L1 antagonist, e.g., antagonist antibody, a CTLA-4 antagonist, e.g., antagonist antibody and/or a LAG3 antagonist, e.g., an antagonist antibody, such that an immune response is stimulated in the subject, for example to inhibit tumor growth or to stimulate an anti-viral response. In one embodiment, the subject is administered an agonist anti-huCD40 antibody and an antagonist anti-PD-1 antibody. In one embodiment, the subject is administered an agonist anti-huCD40 antibody and an antagonist anti-PD-L1 antibody. In one embodiment, the subject is administered an agonist anti-huCD40 antibody and an antagonist anti-CTLA-4 antibody. In one embodiment, the at least one additional immunostimulatory antibody (e.g., an antagonist anti-PD-1, an antagonist anti-PD-L1, an antagonist anti-CTLA-4 and/or an antagonist anti-LAG3 antibody) is a human antibody. Alternatively, the at least one additional immunostimulatory antibody can be, for example, a chimeric or humanized antibody (e.g., prepared from a mouse anti-PD-1, anti-PD-L1, anti-CTLA-4 and/or anti-LAG3 antibody).

Provided herein are methods for treating a hyperproliferative disease (e.g., cancer), comprising administering an agonist anti-huCD40 antibody and an antagonist PD-1 antibody to a subject. In certain embodiments, the agonist anti-huCD40 antibody is administered at a subtherapeutic dose, the anti-PD-1 antibody is administered at a subtherapeutic dose, or both are administered at a subtherapeutic dose. Also provided herein are methods for altering an adverse event associated with treatment of a hyperproliferative disease with an immunostimulatory agent, comprising administering an agonist anti-huCD40 antibody and a subtherapeutic dose of anti-PD-1 antibody to a subject. In certain embodiments, the subject is human. In certain embodiments, the anti-PD-1 antibody is a human sequence monoclonal antibody and the agonist anti-huCD40 antibody is a humanized monoclonal antibody, such as an antibody comprising the CDRs or variable regions of the antibodies disclosed herein.

Suitable PD-1 antagonists for use in the methods described herein, include, without limitation, ligands, antibodies (e.g., monoclonal antibodies and bispecific antibodies), and multivalent agents. In one embodiment, the PD-1 antagonist is a fusion protein, e.g., an Fc fusion protein, such as AMP-244. In one embodiment, the PD-1 antagonist is an anti-PD-1 or anti-PD-L1 antibody.

An exemplary anti-PD-1 antibody is OPDIVO®/nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (KEYTRUDA®/pembrolizumab/formerly lambrolizumab) described in WO2012/145493; AMP-514/MEDI-0680 described in WO 2012/145493; and CT-011 (pidilizumab; previously CT-AcTibody or BAT; see, e.g., Rosenblatt et al. (2011) J. Immunotherapy 34:409). Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO2013/173223 may also be used. An anti-PD-1 antibody that competes for binding with, and/or binds to the same epitope on PD-1 as, as one of these antibodies may also be used in combination treatments.

In certain embodiments, the anti-PD-1 antibody binds to human PD-1 with a K_{D} of 5×10⁻⁸M or less, binds to human PD-1 with a K_{D} of 1×10⁻⁸M or less, binds to human PD-1 with a K_{D} of 5×10⁻⁹M or less, or binds to human PD-1 with a K_{D} of between 1×10⁻⁸M and 1×10⁻¹⁰ M or less.

Provided herein are methods for treating a hyperproliferative disease (e.g., cancer), comprising administering an agonist anti-huCD40 antibody and an antagonist PD-L1 antibody to a subject. In certain embodiments, the agonist anti-huCD40 antibody is administered at a subtherapeutic dose, the anti-PD-L1 antibody is administered at a subtherapeutic dose, or both are administered at a subtherapeutic dose. Provided herein are methods for altering an adverse event associated with treatment of a hyperproliferative disease with an immunostimulatory agent, comprising administering an agonist anti-huCD40 antibody and a subtherapeutic dose of anti-PD-L1 antibody to a subject. In certain embodiments, the subject is human. In certain embodiments, the anti-PD-L1 antibody is a human sequence monoclonal antibody and the agonist anti-huCD40 antibody is a humanized monoclonal antibody, such as an antibody comprising the CDRs or variable regions of the antibodies disclosed herein.

In one embodiment, the anti-PD-L1 antibody is BMS-936559 (referred to as 12A4 in WO 2007/005874 and U.S. Pat. No. 7,943,743), MSB0010718C (WO2013/79174), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MEDI4736 (also known as Anti-B7-H1) or MPDL3280A (also known as RG7446). Any of the anti-PD-L1 antibodies disclosed in WO2013/173223, WO2011/066389, WO2012/145493, U.S. Pat. Nos. 7,635,757 and 8,217,149 and U.S. Publication No. 2009/145493 may also be used. Anti-PD-L1 antibodies that compete with and/or bind to the same epitope as that of any of these antibodies may also be used in combination treatments.

In yet further embodiment, the agonist anti-huCD40 antibody of the present invention is combined with an antagonist of PD-1/PD-L1 signaling, such as a PD-1 antagonist or a PD-L1 antagonist, in combination with a third immunotherapeutic agent. In one embodiment the third immunotherapeutic agent is a GITR antagonist or an OX-40 antagonist, such as the anti-GITR or anti-OX40 antibodies disclosed herein.

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO09/1156652, WO11/56652, WO12/142237).

Provided herein are methods for treating a hyperproliferative disease (e.g., cancer), comprising administering an agonist anti-huCD40 antibody described herein and a CTLA-4 antagonist antibody to a subject. In certain embodiments, the agonist anti-huCD40 antibody is administered at a subtherapeutic dose, the anti-CTLA-4 antibody is administered at a subtherapeutic dose, or both are administered at a subtherapeutic dose. Provided herein are methods for altering an adverse event associated with treatment of a hyperproliferative disease with an immunostimulatory agent, comprising administering an agonist anti-huCD40 antibody and a subtherapeutic dose of anti-CTLA-4 antibody to a subject. In certain embodiments, the subject is human. In certain embodiments, the anti-CTLA-4 antibody is an antibody selected from the group consisting of: YERVOY® (ipilimumab or antibody 10D1, described in PCT Publication WO 01/14424), tremelimumab (formerly ticilimumab, CP-675,206), and the anti-CTLA-4 antibodies described in the following publications: WO 98/42752; WO 00/37504; U.S. Pat. No. 6,207,156; Hurwitz et al. (1998) Proc. Natl. Acad. Sci. USA 95(17):10067-10071; Camacho et al. (2004) J. Clin. Oncology 22(145): Abstract No. 2505 (antibody CP-675206); and Mokyr et al. (1998) Cancer Res. 58:5301-5304. Any of the anti-CTLA-4 antibodies disclosed in WO2013/173223 may also be used.

Provided herein are methods for treating a hyperproliferative disease (e.g., cancer), comprising administering an agonist anti-huCD40 antibody and an anti-LAG-3 antibody to a subject. In further embodiments, the agonist anti-huCD40 antibody is administered at a subtherapeutic dose, the anti-LAG-3 antibody is administered at a subtherapeutic dose, or both are administered at a subtherapeutic dose. Provided herein are methods for altering an adverse event associated with treatment of a hyperproliferative disease with an immunostimulatory agent, comprising administering an agonist anti-huCD40 antibody and a subtherapeutic dose of anti-LAG-3 antibody to a subject. In certain embodiments, the subject is human. In certain embodiments, the anti-LAG-3 antibody is a human sequence monoclonal antibody and the agonist anti-huCD40 antibody is a humanized monoclonal antibody, such as an antibody comprising the CDRs or variable regions of the antibodies disclosed herein. Examples of anti-LAG3 antibodies include antibodies comprising the CDRs or variable regions of antibodies 25F7, 26H10, 25E3, 8B7, 11F2 or 17E5, which are described in U.S. Patent Publication No. US2011/0150892 and WO2014/008218. In one embodiment, an anti-LAG-3 antibody is BMS-986016. Other art recognized anti-LAG-3 antibodies that can be used include IMP731 described in US 2011/007023. IMP-321 may also be used. Anti-LAG-3 antibodies that compete with and/or bind to the same epitope as that of any of these antibodies may also be used in combination treatments.

In certain embodiments, the anti-LAG-3 antibody binds to human LAG-3 with a K_{D} of 5×10⁻⁸ M or less, binds to human LAG-3 with a K_{D} of 1×10⁻⁸ M or less, binds to human LAG-3 with a K_{D} of 5×10⁻⁹M or less, or binds to human LAG-3 with a K_{D} of between 1×10⁻⁸M and 1×10⁻¹⁰ M or less.

Administration of agonist anti-huCD40 antibodies described herein and antagonists, e.g., antagonist antibodies, to one or more second target antigens such as LAG-3 and/or CTLA-4 and/or PD-1 and/or PD-L1 can enhance the immune response to cancerous cells in the patient. Cancers whose growth may be inhibited using the antibodies of the instant disclosure include cancers typically responsive to immunotherapy. Representative examples of cancers for treatment with the combination therapy of the instant disclosure include those cancers specifically listed above in the discussion of monotherapy with agonist anti-huCD40 antibodies.

In certain embodiments, the combination of therapeutic antibodies discussed herein can be administered concurrently as a single composition in a pharmaceutically acceptable carrier, or concurrently as separate compositions with each antibody in a pharmaceutically acceptable carrier. In another embodiment, the combination of therapeutic antibodies can be administered sequentially. For example, an anti-CTLA-4 antibody and an agonist anti-huCD40 antibody can be administered sequentially, such as anti-CTLA-4 antibody being administered first and agonist anti-huCD40 antibody second, or agonist anti-huCD40 antibody being administered first and anti-CTLA-4 antibody second. Additionally or alternatively, an anti-PD-1 antibody and an agonist anti-huCD40 antibody can be administered sequentially, such as anti-PD-1 antibody being administered first and agonist anti-huCD40 antibody second, or agonist anti-huCD40 antibody being administered first and anti-PD-1 antibody second. Additionally or alternatively, an anti-PD-L1 antibody and an agonist anti-huCD40 antibody can be administered sequentially, such as anti-PD-L1 antibody being administered first and agonist anti-huCD40 antibody second, or agonist anti-huCD40 antibody being administered first and anti-PD-L1 antibody second. Additionally or alternatively, an anti-LAG-3 antibody and an agonist anti-huCD40 antibody can be administered sequentially, such as anti-LAG-3 antibody being administered first and agonist anti-huCD40 antibody second, or agonist anti-huCD40 antibody being administered first and anti-LAG-3 antibody second.

Furthermore, if more than one dose of the combination therapy is administered sequentially, the order of the sequential administration can be reversed or kept in the same order at each time point of administration, sequential administrations can be combined with concurrent administrations, or any combination thereof. For example, the first administration of a combination anti-CTLA-4 antibody and agonist anti-huCD40 antibody can be concurrent, the second administration can be sequential with anti-CTLA-4 antibody first and agonist anti-huCD40 antibody second, and the third administration can be sequential with agonist anti-huCD40 antibody first and anti-CTLA-4 antibody second, etc. Additionally or alternatively, the first administration of a combination anti-PD-1 antibody and agonist anti-huCD40 antibody can be concurrent, the second administration can be sequential with anti-PD-1 antibody first and agonist anti-huCD40 antibody second, and the third administration can be sequential with agonist anti-huCD40 antibody first and anti-PD-1 antibody second, etc. Additionally or alternatively, the first administration of a combination anti-PD-L1 antibody and agonist anti-huCD40 antibody can be concurrent, the second administration can be sequential with anti-PD-L1 antibody first and agonist anti-huCD40 antibody second, and the third administration can be sequential with agonist anti-huCD40 antibody first and anti-PD-L1 antibody second, etc. Additionally or alternatively, the first administration of a combination anti-LAG-3 antibody and agonist anti-huCD40 antibody can be concurrent, the second administration can be sequential with anti-LAG-3 antibody first and agonist anti-huCD40 antibody second, and the third administration can be sequential with agonist anti-huCD40 antibody first and anti-LAG-3 antibody second, etc. Another representative dosing scheme can involve a first administration that is sequential with agonist anti-huCD40 first and anti-CTLA-4 antibody (and/or anti-PD-1 antibody and/or anti-PD-L1 antibody and/or anti-LAG-3 antibody) second, and subsequent administrations may be concurrent.

Optionally, an agonist anti-huCD40 as sole immunotherapeutic agent, or the combination of an agonist anti-huCD40 antibody and one or more additional immunotherapeutic antibodies (e.g., anti-CTLA-4 and/or anti-PD-1 and/or anti-PD-L1 and/or anti-LAG-3 blockade) can be further combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below). A CD40 agonist and one or more additional antibodies (e.g., CTLA-4 and/or PD-1 and/or PD-L1 and/or LAG-3 blockade) can also be further combined with standard cancer treatments. For example, a CD40 agonist and one or more additional antibodies (e.g., CTLA-4 and/or PD-1 and/or PD-L1 and/or LAG-3 blockade) can be effectively combined with chemotherapeutic regimes. In these instances, it is possible to reduce the dose of other chemotherapeutic reagent administered with the combination of the instant disclosure (Mokyr et al. (1998) Cancer Research 58: 5301-5304). An example of such a combination is a combination of CD40 agonist antibody with or without and an additional antibody, such as anti-CTLA-4 antibodies and/or anti-PD-1 antibodies and/or anti-PD-L1 antibodies and/or anti-LAG-3 antibodies) further in combination with decarbazine for the treatment of melanoma. Another example is a combination of agonist anti-huCD40 antibody with or without and anti-CTLA-4 antibodies and/or anti-PD-1 antibodies and/or anti-PD-L1 antibodies and/or LAG-3 antibodies further in combination with interleukin-2 (IL-2) for the treatment of melanoma. The scientific rationale behind the combined use of CD40 agonism and CTLA-4 and/or PD-1 and/or PD-L1 and/or LAG-3 blockade with chemotherapy is that cell death, which is a consequence of the cytotoxic action of most chemotherapeutic compounds, should result in increased levels of tumor antigen in the antigen presentation pathway. Other combination therapies that may result in synergy with a combined CD40 agonism with or without and CTLA-4 and/or PD-1 and/or PD-L1 and/or LAG-3 blockade through cell death include radiation, surgery, or hormone deprivation. Each of these protocols creates a source of tumor antigen in the host. Angiogenesis inhibitors can also be combined with a combined CD40 agonism and CTLA-4 and/or PD-1 and/or PD-L1 and/or LAG-3 blockade. Inhibition of angiogenesis leads to tumor cell death, which can be a source of tumor antigen fed into host antigen presentation pathways.

An agonist anti-huCD40 antibody as sole immunotherapeutic agent, or a combination of CD40 agonist and CTLA-4 and/or PD-1 and/or PD-L1 and/or LAG-3 blocking antibodies can also be used in combination with bispecific antibodies that target Fcα or Fcγ receptor-expressing effector cells to tumor cells. See, e.g., U.S. Pat. Nos. 5,922,845 and 5,837,243. Bispecific antibodies can be used to target two separate antigens. The T cell arm of these responses would be augmented by the use of a combined CD40 agonism and CTLA-4 and/or PD-1 and/or PD-L1 and/or LAG-3 blockade.

In another example, an agonistic anti-CD40 antibody as sole immunotherapeutic agent or a combination of an anti-CD40 antibody and additional immunostimulating agent, e.g., anti-CTLA-4 antibody and/or anti-PD-1 antibody and/or anti-PD-L1 antibody and/or LAG-3 agent, e.g., antibody, can be used in conjunction with an anti-neoplastic antibody, such as RITUXAN® (rituximab), HERCEPTIN® (trastuzumab), BEXXAR® (tositumomab), ZEVALIN® (ibritumomab), CAMPATH® (alemtuzumab), LYMPHOCIDE® (eprtuzumab), AVASTIN® (bevacizumab), and TARCEVA® (erlotinib), and the like. By way of example and not wishing to be bound by theory, treatment with an anti-cancer antibody or an anti-cancer antibody conjugated to a toxin can lead to cancer cell death (e.g., tumor cells) which would potentiate an immune response mediated by the immunostimulating agent, e.g., CD40, TIGIT, CTLA-4, PD-1, PD-L1 or LAG-3 agent, e.g., antibody. In an exemplary embodiment, a treatment of a hyperproliferative disease (e.g., a cancer tumor) can include an anti-cancer agent, e.g., antibody, in combination with an agonist anti-huCD40 antibody and optionally an additional immunostimulating agent, e.g., anti-CTLA-4 and/or anti-PD-1 and/or anti-PD-L1 and/or anti-LAG-3 agent, e.g., antibody, concurrently or sequentially or any combination thereof, which can potentiate an anti-tumor immune responses by the host.

Provided herein are methods for altering an adverse event associated with treatment of a hyperproliferative disease (e.g., cancer) with an immunostimulatory agent, comprising administering an agonist anti-huCD40 antibody with or without and a subtherapeutic dose of anti-CTLA-4 and/or anti-PD-1 and/or anti-PD-L1 and/or anti-LAG-3 agent, e.g., antibody, to a subject. For example, the methods described herein provide for a method of reducing the incidence of immunostimulatory therapeutic antibody-induced colitis or diarrhea by administering a non-absorbable steroid to the patient. As used herein, a “non-absorbable steroid” is a glucocorticoid that exhibits extensive first pass metabolism such that, following metabolism in the liver, the bioavailability of the steroid is low, i.e., less than about 20%. In one embodiment described herein, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. The usual oral dosage of ENTOCORT EC® for the treatment of Crohn's disease is 6 to 9 mg/day. ENTOCORT EC® is released in the intestines before being absorbed and retained in the gut mucosa. Once it passes through the gut mucosa target tissue, ENTOCORT EC® is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT EC® can result in systemic glucocorticoid effects such as hypercorticism and adrenal suppression. See PDR 58^{th} ed. 2004; 608-610.

In still further embodiments, a CD40 agonist with or without CTLA-4 and/or PD-1 and/or PD-L1 and/or LAG-3 blockade (i.e., immunostimulatory therapeutic antibodies against CD40 and optionally anti-CTLA-4 and/or anti-PD-1 and/or anti-PD-L1 and/or anti-LAG-3 antibodies) in conjunction with a non-absorbable steroid can be further combined with a salicylate. Salicylates include 5-ASA agents such as, for example: sulfasalazine (AZULFIDINE®, Pharmacia & UpJohn); olsalazine (DIPENTUM®, Pharmacia & UpJohn); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay).

In accordance with the methods described herein, a salicylate administered in combination with agonist anti-huCD40 antibody with or without anti-CTLA-4 and/or anti-PD-1 and/or anti-PD-L1 and/or LAG-3 antibodies and a non-absorbable steroid can includes any overlapping or sequential administration of the salicylate and the non-absorbable steroid for the purpose of decreasing the incidence of colitis induced by the immunostimulatory antibodies. Thus, for example, methods for reducing the incidence of colitis induced by the immunostimulatory antibodies described herein encompass administering a salicylate and a non-absorbable concurrently or sequentially (e.g., a salicylate is administered 6 hours after a non-absorbable steroid), or any combination thereof. Further, a salicylate and a non-absorbable steroid can be administered by the same route (e.g., both are administered orally) or by different routes (e.g., a salicylate is administered orally and a non-absorbable steroid is administered rectally), which may differ from the route(s) used to administer the anti-huCD40 and anti-CTLA-4 and/or anti-PD-1 and/or anti-PD-L1 and/or anti-LAG-3 antibodies.

The agonist anti-huCD40 antibodies and combination antibody therapies described herein may also be used in conjunction with other well-known therapies that are selected for their particular usefulness against the indication being treated (e.g., cancer). Combinations of the agonist anti-huCD40 antibodies described herein may be used sequentially with known pharmaceutically acceptable agent(s).

For example, the agonist anti-huCD40 antibodies and combination antibody therapies described herein can be used in combination (e.g., simultaneously or separately) with an additional treatment, such as irradiation, chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo2l/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 (IDO1) inhibitor (e.g., INCB24360), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., AVASTIN®), synthetic triterpenoids (see Hyer et al., Cancer Research 2005; 65:4799-808), c-FLIP (cellular FLICE-inhibitory protein) modulators (e.g., natural and synthetic ligands of PPARγ (peroxisome proliferator-activated receptor γ), 5809354 or 5569100), kinase inhibitors (e.g., Sorafenib), trastuzumab, cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3β inhibitors, IAP inhibitors and/or genotoxic drugs.

The agonist anti-huCD40 antibodies and combination antibody therapies described herein can further be used in combination with one or more anti-proliferative cytotoxic agents. Classes of compounds that may be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with agonist anti-huCD40 antibodies, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bl, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with agonist anti-huCD40 antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX′, can also be administered to the patient. When employing the methods or compositions described herein, other agents used in the modulation of tumor growth or metastasis in a clinical setting, such as antimimetics, can also be administered as desired.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### IX. Characterization of Specific Agonist Anti-CD40 Antibodies of the Present Invention

Agonist anti-CD40 antibodies of the present invention were obtained as described in WO 2017/004006. Variable domains and CDR sequence regions of exemplary antibodies are provided in the Sequence Listing, and are summarized at Table 7. Variable domain and CDR region numbering is the same for all antibodies derived from the same original clone, i.e. the humanized variants provided herein do not include any insertions or deletions.

TABLE 7
Antibody Variable Domains and CDRs

| Clone | Chain | Variable Domain | CDR1 | CDR2 | CDR3 |
|---|---|---|---|---|---|
| 12D6 | Heavy chain | 1-119 | 31-35 | 50-66 | 99-108 |
| 12D6 | Light chain | 1-112 | 24-39 | 55-61 | 94-102 |
| 5F11 | Heavy chain | 1-117 | 31-35 | 50-66 | 99-106 |
| 5F11 | Light chain | 1-111 | 24-38 | 54-60 | 93-101 |
| 8E8 | Heavy chain | 1-122 | 31-35 | 50-66 | 99-111 |
| 8E8 | Light chain | 1-112 | 24-39 | 55-61 | 94-102 |
| 5G7 | Heavy chain | 1-113 | 31-35 | 50-66 | 99-102 |
| 5G7 | Light chain | 1-107 | 24-34 | 50-56 | 89-97 |
| 19G3 | Heavy chain | 1-112 | 31-35 | 50-66 | 99-101 |
| 19G3 | Light chain | 1-112 | 24-39 | 55-61 | 94-102 |

The present disclosure is further illustrated by the following examples, which should not be construed as further limiting. The contents of all figures and all references, Genbank sequences, patents and published patent applications cited throughout this application are expressly incorporated herein by reference.

### Example 1

Characterization of Altered Heavy Chain Antibodies for Reduced Effector Function

The binding of human FcγRs to antibodies was studied by surface plasmon resonance using a Biacore 8K system (GE Healthcare, USA). For these studies, protein A was immobilized on flow cells 1-4 of the CM5 sensor chip using standard ethyl (dimethylaminopropyl) carbodiimide (EDC)/N-hydroxysuccinimide (NETS) chemistry, with ethanolamine blocking, in a running buffer of 10 mM HEPES pH 7.4, 150 mM NaCl, 3 mM EDTA, 0.05% surfactant p20, to a density of ˜3000 RU. Purified 12D6-24-IgG1f antibody (10 μg/mL) or expression supernatants of all other antibodies (diluted to ˜10 μg/ml) were captured on the protein A surface to a density of ˜1000-1200 RU, and the binding of FcγR analytes was tested in running buffer consisting of 10 mM NaPO₄, 130 mM NaCl, 0.05% p20, buffer (PBS-T) pH 7.1 at 25° C., using 120 s association time and 120 s dissociation time at a flow rate of 20 μL/min. The data were analyzed using Biacore 8K evaluation software by determining the measured binding response as a percentage of theoretical maximum binding response for each antibody (% Rmax), based on the level of captured antibody, assuming 100% fractional activity and only taking into account protein mass without glycosylation. Results are provided at Table 8.

TABLE 8
Binding of Anti-CD40 12D6-24 Agonist Antibody
Fc Variants to Human Fc Receptors

| | | 10 μM | 10 μM | | 10 μM | 10 μM |
|---|---|---|---|---|---|---|
| | 1 μM | hCD32a- | hCD32a- | 10 μM | hCD16a- | hCD16a- |
| Antibody | hCD64 | H131 | R131 | hCD32b | V158 | F158 |
| 12D6-24-IgG1f | 123% | 98% | 96% | 73% | 116% | 65% |
| 12D6-IgG1f-P238K | 109% | −3% | −2% | −2% | −4% | −4% |
| 12D6-IgG1f-L235E- | 11% | 1% | 3% | 3% | 0% | −1% |
| P238K | | | | | | |
| 12D6-IgG1f-L235E- | 11% | 2% | 5% | 3% | 0% | 0% |
| P238K-K322A | | | | | | |
| 12D6-IgG2.3G1- | 16% | 1% | 4% | 3% | 1% | 0% |
| L235E-P238K | | | | | | |
| 12D6-IgG2.3G1- | 16% | 2% | 5% | 3% | 0% | 0% |
| L235E-P238K-K322A | | | | | | |
| 12D6-IgG2.5G1- | 15% | 2% | 5% | 3% | 0% | 0% |
| L235E-P238K-K322A | | | | | | |
| 12D6-IgG1.3f-P238K | 1% | 1% | 3% | 2% | 0% | 0% |
| 12D6-IgG2.3G1.3f- | 2% | 1% | 3% | 1% | 0% | 0% |
| P238K | | | | | | |
| 12D6-IgG2.5G1.3f- | 2% | 1% | 3% | 2% | 0% | 0% |
| P238K | | | | | | |

The P238K mutation dramatically reduces binding to all Fc receptors tested except CD64 (FcγRI). Addition of the L235E mutation to P238K reduces CD64 binding ˜10-fold. Addition of the IgG1.3f variant (L234A, L235E and G237A) to P238K effectively eliminates binding to CD64, leaving constructs with little to no binding to any of the Fc receptors tested. Further addition of K322A does not significantly affect FcγR binding. Substitution of the CH1 and upper hinge regions of either IgG2.3 or IgG2.5 into the IgG1f and IgG1.3f constructs also had little effect on FcγR binding, meaning that IgG2.3 and IgG2.5 constructs of the present invention may be combined with the mutation(s) recited in Table 8 to create enhanced agonist anti-CD40 antibodies with reduced or eliminated effector function.

TABLE 9
Summary of Sequence Listing

| SEQ ID: | Description |
|---|---|
| 1 | Human CD40 (NP_001241) |
| 2 | Human CD40L-gp39 (NP_000065.1) |
| 3 | 12D6 Chimeric Heavy Chain |
| 4 | 12D6 Chimeric Light Chain |
| 5 | 12D6-03 Heavy Chain |
| 6 | 12D6-03 Light Chain |
| 7 | 12D6-22 Heavy Chain |
| 8 | 12D6-22/12D6-24 Light Chain |
| 9 | 12D6-23 Heavy Chain |
| 10 | 12D6-23 Light Chain |
| 11 | 12D6-24 Heavy Chain |
| 2 | 5F11 Chimeric Heavy Chain |
| 13 | 5F11 Chimeric Light Chain |
| 14 | 5F11-17 Heavy Chain |
| 15 | 5F11-17 Light Chain |
| 16 | 5F11-23 Heavy Chain |
| 17 | 5F11-23 Light Chain |
| 18 | 5F11-45 Heavy Chain |
| 19 | 5F11-45 Light Chain |
| 20 | 8E8 Chimeric Heavy Chain |
| 21 | 8E8 Chimeric Light Chain |
| 22 | 8E8-56 Heavy Chain |
| 23 | 8E8-56 Light Chain |
| 24 | 8E8-62 Heavy Chain |
| 25 | 8E8-62 Light Chain |
| 26 | 8E8-67 Heavy Chain |
| 27 | 8E8-67 Light Chain |
| 28 | 8E8-70 Heavy Chain |
| 29 | 8E8-70 Light Chain |
| 30 | 8E8-71 Heavy Chain |
| 31 | 8E8-71 Light Chain |
| 32 | 5G7 Chimeric Heavy Chain |
| 33 | 5G7 Chimeric Light Chain |
| 34 | 5G7-22 Heavy Chain |
| 35 | 5G7-22 Light Chain |
| 36 | 5G7-25 Heavy Chain |
| 37 | 5G7-25 Light Chain |
| 38 | 19G3 Chimeric Heavy Chain |
| 39 | 19G3 Chimeric Light Chain |
| 40 | 19G3-11 Heavy Chain |
| 41 | 19G3-11 Light Chain |
| 42 | 19G3-22 Heavy Chain |
| 43 | 19G3-22 Light Chain |
| 44 | Human Constant Region IgG1f |
| 45 | Light Chain Kappa Constant Region |
| 46 | Signal Sequence |
| 47 | IgG1 C-terminal CH1 (same for IgG3 (17-15-15-15), |
| | igG3 (17-15-15), IgG3 (17-15), IgG3 (15-15-15), |
| | IgG3 (15), and IgG4 |
| 48 | IgG1 upper hinge |
| 49 | IgG1 middle hinge |
| 50 | IgG1 lower hinge (same for IgG3 (17-15-15-15), |
| | IgG3 (17-15-15), IgG3 (17-15), IgG3 (15-15-15), |
| | IgG3 (15), and IgG4) |
| 51 | IgG2 C-terminal CH1 |
| 52 | IgG2 middle hinge |
| 53 | IgG2 lower hinge |
| 54 | IgG3 (17-15-15-15) upper hinge (same for IgG3 |
| | (17-15-15) and IgG3 (17-15)) |
| 55 | IgG3 (17-15-15-15) middle hinge |
| 56 | IgG3 (17-15-15) middle hinge |
| 57 | IgG3 (17-15) middle hinge |
| 58 | IgG3 (15-15-15) upper hinge (same for IgG3(15)) |
| 59 | IgG3 (15-15-15) middle hinge |
| 60 | IgG3 (15) middle hinge |
| 61 | IgG4 upper hinge |
| 62 | IgG4 middle hinge |
| 63 | IgG4 lower hinge |
| 64 | Wildtype human IgG1 CH1 |
| 65 | Wildtype human IgG2 CH1 |
| 66 | Wildtype human IgG1 CH2 |
| 67 | Human IgG1 CH2 with A330S/P331S |
| 68 | Wildtype human IgG1 CH3 |
| 69 | IgG1-IgG2-IgG1f |
| 70 | IgG1-IgG2CS-IgG1f |
| 71 | IgG1-IgG2-IgG1.1f |
| 72 | IgG1-IgG2CS-IgG1.1f |
| 73 | IgG1-IgG2-1gG1f2 |
| 74 | IgG1-IgG2(C219S)-IgG1f2 |
| 75 | IgG2-IgG1f2 |
| 76 | IgG2(C219S)-IgG1f2 |
| 77 | WT human IgG2 hinge |
| 78 | Human IgG2 hinge with C219S |
| 79 | IgG2/IgG1 hinge |
| 80 | IgG2 (C219S)/IgG1 hinge |
| 81 | Wild type human IgG1 hinge |
| 82 | IgG1.1 Hinge (L234A/L235E/G237A) |
| 83 | Wildtype human IgG2 CH2 |
| 84 | Wildtype human IgG2 CH3 |
| 85 | IgG1f with C-terminal K |
| 86 | IgG2.3 |
| 87 | IgG2.3G1-AY |
| 88 | IgG2.3G1-KH |
| 89 | IgG2.5 |
| 90 | IgG1.1f |
| 91 | IgG2.3G1.1f-KH |
| 92 | IgG1-deltaTHT |
| 93 | IgG2.3-plusTHT |
| 94 | IgG2.3-plusGGG |
| 95 | IgG2.5G1.1f-KH |
| 96 | IgG2.5G1-AY |
| 97 | IgG2.5G1-KH |
| 98 | IgG2.5-plusTHT |
| 99 | IgG1-G2.3G1-AY |
| 100 | IgG1-G2.3G1-KH |
| 101 | G2-G1-G1-G1 |
| 102 | G2.5-G1-G1-G1 |
| 103 | G1-G2.3-G2-G2 |
| 104 | G1-KRGEGSSNLF |
| 105 | G1-KRGEGS |
| 106 | G1-SNLF |
| 107 | IgG1-ITNDRTPR |
| 108 | G1-SNLFPR |
| 109 | G2-RKEGSGNSFL |
| 110 | G2-RKEGSG |
| 111 | G2-NSFL |
| 112 | IgG2-TIDNTRRP |
| 113 | G2-NSFLRP |
| 114 | G1-G1-G2-G1-AY |
| 115 | G1-G1-G2-G1-KH |
| 116 | G2-G2.3-G1-G2-KH |
| 117 | G2.5-G2.3-G1-G2-KH |
| 118 | G2-G2.3-G1-G2-AY |
| 119 | G2.5-G2.3-G1-G2-AY |
| 120 | G1-G2.3-G1-G1-KH |
| 121 | G2-G1-G2-G2-AY |
| 122 | G2.5-G1-G2-G2-AY |
| 123 | G1-G2-G1-G1-AY |
| 124 | G2-G1-G2-G2-KH |
| 125 | G2.5-G1-G2-G2-KH |
| 126 | IgG1-deltaHinge |
| 127 | IgG2-deltaHinge |
| 128 | IgG2.5-deltaHinge |
| 129 | IgG1-deltaG237 |
| 130 | IgG2-plusG237 |
| 131 | IgG2.4 |
| 132 | IgG2.3/4 |
| 133 | Hinge IgG2 C220S |
| 134 | IgG2/IgG1 hybrid hinge C220S |
| 135 | Wildtype IgG2 hinge portion |
| 136 | IgG2 hinge portion C219S |
| 137 | IgG2 hinge portion C220S |
| 138 | IgG2 hinge portion C219X |
| 139 | IgG2 hinge portion C220X |
| 140 | IgG2 CH1 + IgG2 hinge (wildtype) |
| 141 | IgG2 with C219X |
| 142 | IgG2 with C220X |
| 143 | IgG2/IgG1 hybrid with C219X |
| 144 | IgG2/IgG1 hybrid with C220X |
| 145 | IgG2/IgG1 hybrid deltaG |
| 146 | IgG2/IgG1 hybrid with C219S deltaG |
| 147 | IgG2/IgG1 hybrid with C220S deltaG |
| 148 | IgG2/IgG1 hybrid with C219X deltaG |
| 149 | IgG2/IgG1 hybrid with C220X deltaG |
| 150 | IgG2 hinge portion |
| 151 | IgG1 hinge portion |
| 152 | IgG1 hinge portion 1 |
| 153 | IgG1 hinge portion 2 |
| 154 | IgG1f P238K |
| 155 | IgG1.3f |
| 156 | IgG1.3f P238K |
| 157 | IgG1f L235E P238K |
| 158 | IgG1f L235E P238K K322A |
| 159 | IgG2.3 P238K |
| 160 | IgG2.3/IgG1 hybrid P238K |
| 161 | IgG2.3/IgG1.3f hybrid |
| 162 | IgG2.3/IgG1.3f hybrid P238K |
| 163 | IgG2.3/IgG1 hybrid L235E P238K |
| 164 | IgG2.3/IgG1 hybrid L235E P238K K322A |
| 165 | IgG2.5 P238K |
| 166 | IgG2.5/IgG1 hybrid P238K |
| 167 | IgG2.5/IgG1.3f hybrid |
| 168 | IgG2.5/IgG1.3f hybrid P238K |
| 169 | IgG2.5/IgG1 hybrid L235E P238K |
| 170 | IgG2.5/IgG1 hybrid L235E P238K K322A |
| 171 | IgG1f E345K |
| 172 | IgG1f E345R |
| 173 | IgG1f E345R E430G S440Y |
| 174 | IgG1.3f E345K |
| 175 | IgG1.3f E345R |
| 176 | IgG1.3f E345R E430G S440Y |
| 177 | IgG1.3f E345R K322A |
| 178 | IgG2.3 E345K |
| 179 | IgG2.5 E345K |
| 180 | IgG2.3 E345R |
| 181 | IgG2.5 E345R |
| 182 | IgG2.3 E345R E430G S440Y |
| 183 | IgG2.5 E345R E430G S440Y |
| 184 | IgG2.3 E345R D265A |
| 185 | IgG2.5 E345R D265A |
| 186 | IgG2 |

The Sequence Listing provides the sequences of the mature heavy and light chains (i.e., sequences do not include signal peptides). A signal sequence for production of the heavy and/or light chains of the antibodies of the present invention, for example in human cells, is provided at SEQ ID NO: 46.

### EQUIVALENTS

Those skilled in the art will recognize, or be able to ascertain using no more than routine experimentation, many equivalents of the specific embodiments disclosed herein. Such equivalents are intended to be encompassed by the following claims.

1. An isolated antibody that specifically binds to human CD40 comprising a heavy chain constant region selected from the group consisting of SEQ ID NOs: 159-170.

2. The isolated antibody of claim 1, wherein the heavy chain constant region is selected from the group consisting of SEQ ID NOs: 159-161 and 165-167.

3. An isolated antibody that specifically binds to human CD40 comprising a heavy chain constant region selected from the group consisting of SEQ ID NOs: 171-185.

4. The isolated antibody of claim 3, wherein the antibody exhibits reduced or eliminated effector function when compared with an otherwise identical antibody comprising a wildtype IgG1f constant region (SEQ ID NO: 44).

5. The isolated antibody of claim 3, wherein the heavy chain constant region is selected from the group consisting of SEQ ID NOs: 174-175.

6. The isolated antibody of claim 5, wherein the heavy chain constant region is SEQ ID NOs: 175.

7. The isolated antibody of claim 1 further comprising an antigen binding domain that competes for binding to human CD40 in a cross-blocking assay with one or more of antibodies selected from the group consisting of 12D6 (SEQ ID NOs: 3 and 4), 5F11 (SEQ ID NOs: 12 and 13), 8E8 (SEQ ID NOs: 20 and 21), 5G7 (SEQ ID NOs: 32 and 33), and 19G3 (SEQ ID NOs: 38 and 39).

8. The isolated antibody of claim 7, wherein the competition in a cross-blocking assay comprises the ability to reduce binding of the selected antibody to human CD40 (SEQ ID NO: 1) in a competition ELISA by at least 20% when used at an equal molar concentration with the selected antibody.

9. The isolated antibody of claim 1 further comprising an antigen binding domain that specifically binds to human CD40 at:
a. an epitope comprising or consisting of the sequence WGCLLTAVHPEPPTACREKQYLINS (residues 11-35 of SEQ ID NO: 1) (antibody 12D6);
b. an epitope comprising or consisting of the sequence EPPTACREKQYLINS (residues 21-35 of SEQ ID NO: 1) (antibody 5G7); or
c. an epitope comprising or consisting of the sequence ECLPCGESE (residues 58-66 of SEQ ID NO: 1) (antibody 5F11).

10. The isolated antibody of claim 1 further comprising an antigen binding domain that specifically binds to human CD40 comprising:
a) heavy chain CDR sequences derived at least in part from murine V region germline VH1-39_01 and J region germline IGHJ4 and
light chain CDR sequences derived at least in part from murine V region germline VK1-110_01 and J region germline IGKJ1 (12D6);
b) heavy chain CDR sequences derived at least in part from murine V region germline VH1-4_02 and J region germline IGHJ3 and
light chain CDR sequences derived at least in part from murine V region germline VK3-5_01 and J region germline IGKJ5 (5F11);
c) heavy chain CDR sequences derived at least in part from murine V region germline VH1-80_01 and J region germline IGHJ2 and
light chain CDR sequences derived at least in part from murine V region germline VK1-110_01 and J region germline IGKJ2 (8E8);
d) heavy chain CDR sequences derived at least in part from murine V region germline VH1-18_01 and J region germline IGHJ4 and
light chain CDR sequences derived at least in part from murine V region germline VK10-96_01 and J region germline IGKJ2 (5G7); or
e) heavy chain CDR sequences derived at least in part from murine V region germline VH5-9-4_01 and J region germline IGHJ3 and
light chain CDR sequences derived at least in part from murine V region germline VK1-117_01 and J region germline IGKJ2 (19G3).

11. The isolated antibody of claim 1 further comprising an antigen binding domain that specifically binds to human CD40, wherein the antibody comprises a heavy chain and a light chain, wherein the heavy chain comprises CDRH1, CDRH2 and CDRH3 sequences and the light chain comprises CDRL1, CDRL2 and CDRL3 sequences selected from the group consisting of:
a) the CDRs of antibody 12D6-03 wherein CDRH1, CDRH2 and CDRH3 comprise residues 31-35, 50-66 and 99-108, respectively, of SEQ ID NO:5 and CDRL1, CDRL2 and CDRL3 comprise residues 24-39, 55-61 and 94-102, respectively, of SEQ ID NO:6;
b) the CDRs of antibody 12D6-22 wherein CDRH1, CDRH2 and CDRH3 comprise residues 31-35, 50-66 and 99-108, respectively, of SEQ ID NO:7 and CDRL1, CDRL2 and CDRL3 comprise residues 24-39, 55-61 and 94-102, respectively, of SEQ ID NO:8;
c) the CDRs of antibody 12D6-23 wherein CDRH1, CDRH2 and CDRH3 comprise residues 31-35, 50-66 and 99-108, respectively, of SEQ ID NO:9 and CDRL1, CDRL2 and CDRL3 comprise residues 24-39, 55-61 and 94-102, respectively, of SEQ ID NO:10;
d) the CDRs of antibody 12D6-24 wherein CDRH1, CDRH2 and CDRH3 comprise residues 31-35, 50-66 and 99-108, respectively, of SEQ ID NO:11 and CDRL1, CDRL2 and CDRL3 comprise residues 24-39, 55-61 and 94-102, respectively, of SEQ ID NO:8;
e) the CDRs of antibody 5F11-17 wherein CDRH1, CDRH2 and CDRH3 comprise residues 31-35, 50-66 and 99-106, respectively, of SEQ ID NO:14 and CDRL1, CDRL2 and CDRL3 comprise residues 24-38, 54-60 and 93-101, respectively, of SEQ ID NO:15;
f) the CDRs of antibody 5F11-23 wherein CDRH1, CDRH2 and CDRH3 comprise residues 31-35, 50-66 and 99-106, respectively, of SEQ ID NO:16 and CDRL1, CDRL2 and CDRL3 comprise residues 24-38, 54-60 and 93-101, respectively, of SEQ ID NO:17;
g) the CDRs of antibody 5F11-45 wherein CDRH1, CDRH2 and CDRH3 comprise residues 31-35, 50-66 and 99-106, respectively, of SEQ ID NO:18 and CDRL1, CDRL2 and CDRL3 comprise residues 24-38, 54-60 and 93-101, respectively, of SEQ ID
h) the CDRs of antibody 8E8-56 wherein CDRH1, CDRH2 and CDRH3 comprise residues 31-35, 50-66 and 99-111, respectively, of SEQ ID NO:22 and CDRL1, CDRL2 and CDRL3 comprise residues 24-39, 55-61 and 94-102, respectively, of SEQ ID NO:23;
i) the CDRs of antibody 8E8-62 wherein CDRH1, CDRH2 and CDRH3 comprise residues 31-35, 50-66 and 99-111, respectively, of SEQ ID NO:24 and CDRL1, CDRL2 and CDRL3 comprise residues 24-39, 55-61 and 94-102, respectively, of SEQ ID NO:25;
j) the CDRs of antibody 8E8-67 wherein CDRH1, CDRH2 and CDRH3 comprise residues 31-35, 50-66 and 99-111, respectively, of SEQ ID NO:26 and CDRL1, CDRL2 and CDRL3 comprise residues 24-39, 55-61 and 94-102, respectively, of SEQ ID NO:27;
k) the CDRs of antibody 8E8-70 wherein CDRH1, CDRH2 and CDRH3 comprise residues 31-35, 50-66 and 99-111, respectively, of SEQ ID NO:28 and CDRL1, CDRL2 and CDRL3 comprise residues 24-39, 55-61 and 94-102, respectively, of SEQ ID NO:29;
l) the CDRs of antibody 8E8-71 wherein CDRH1, CDRH2 and CDRH3 comprise residues 31-35, 50-66 and 99-111, respectively, of SEQ ID NO:30 and CDRL1, CDRL2 and CDRL3 comprise residues 24-39, 55-61 and 94-102, respectively, of SEQ ID NO:31;
m) the CDRs of antibody 5G7-22 wherein CDRH1, CDRH2 and CDRH3 comprise residues 31-35, 50-66 and 99-102, respectively, of SEQ ID NO:34 and CDRL1, CDRL2 and CDRL3 comprise residues 24-34, 50-56 and 89-97, respectively, of SEQ ID NO:35;
n) the CDRs of antibody 5G7-25 wherein CDRH1, CDRH2 and CDRH3 comprise residues 31-35, 50-66 and 99-102, respectively, of SEQ ID NO:36 and CDRL1, CDRL2 and CDRL3 comprise residues 24-34, 50-56 and 89-97, respectively, of SEQ ID NO:37;
o) the CDRs of antibody 19G3-11 wherein CDRH1, CDRH2 and CDRH3 comprise residues 31-35, 50-66 and 99-101, respectively, of SEQ ID NO:40 and CDRL1, CDRL2 and CDRL3 comprise residues 24-39, 55-61 and 94-102, respectively, of SEQ ID NO:41; and
p) the CDRs of antibody 19G3-22 wherein CDRH1, CDRH2 and CDRH3 comprise residues 31-35, 50-66 and 99-101, respectively, of SEQ ID NO:42 and CDRL1, CDRL2 and CDRL3 comprise residues 24-39, 55-61 and 94-102, respectively, of SEQ ID NO:43.

12. The isolated antibody of claim 11 wherein the CDRs comprise the CDRs of antibody 12D6-24 wherein CDRH1, CDRH2 and CDRH3 comprise residues 31-35, 50-66 and 99-108, respectively, of SEQ ID NO:11 and CDRL1, CDRL2 and CDRL3 comprise residues 24-39, 55-61 and 94-102, respectively, of SEQ ID NO:8.

13. The isolated antibody of claim 1 further comprising an antigen binding domain that specifically binds to human CD40, wherein the antibody comprises a heavy chain and a light chain, wherein the heavy chain comprises a heavy chain variable domain sequence selected from the groups consisting of:
a) the heavy chain variable region of antibody 12D6-03 comprising residues 1-119 of SEQ ID NO:5;
b) the heavy chain variable region of antibody 12D6-22 comprising residues 1-119 of SEQ ID NO:7;
c) the heavy chain variable region of antibody 12D6-23 comprising residues 1-119 of SEQ ID NO:9;
d) the heavy chain variable region of antibody 12D6-24 comprising residues 1-119 of SEQ ID NO:11, respectively;
e) the heavy chain variable region of antibody 5F11-17 comprising residues 1-117 of SEQ ID NO:14;
f) the heavy chain variable region of antibody 5F11-23 comprising residues 1-117 of SEQ ID NO:16;
g) the heavy chain variable region of antibody 5F11-45 comprising residues 1-117 of SEQ ID NO:18;
h) the heavy chain variable region of antibody 8E8-56 comprising residues 1-122 of SEQ ID NO:22;
i) the heavy chain variable region of antibody 8E8-62 comprising residues 1-122 of SEQ ID NO:24;
j) the heavy chain variable region of antibody 8E8-67 comprising residues 1-122 of SEQ ID NO:26;
k) the heavy chain variable region of antibody 8E8-70 comprising residues 1-122 of SEQ ID NO:25;
l) the heavy chain variable region of antibody 8E8-71 comprising residues 1-122 of SEQ ID NO:30;
m) the heavy chain variable region of antibody 5G7-22 comprising residues 1-113 of SEQ ID NO:34;
n) the heavy chain variable region of antibody 5G7-25 comprising residues 1-113 of SEQ ID NO:36;
o) the heavy chain variable region of antibody 19G3-11 comprising residues 1-112 of SEQ ID NO:40; and
p) the heavy chain variable region of antibody 19G3-22 comprising residues 1-112 of SEQ ID NO:42.

14. The isolated antibody of claim 13 wherein the heavy chain comprises the heavy chain variable region of antibody 12D6-24 comprising residues 1-119 of SEQ ID NO:11.

15. A nucleic acid molecule encoding the heavy and/or light chain variable region of the antibody of claim 1.

16. An expression vector comprising the nucleic acid molecule of claim 15.

17. A cell transformed with an expression vector of claim 16.

18. A method of preparing an anti-human CD40 antibody comprising:
a) expressing the antibody, or antigen binding portion thereof, in the cell of claim 17; and
b) isolating the antibody from the cell.

19. A pharmaceutical composition comprising:
a) antibody of claim 1; and
b) a carrier.

20. A method of stimulating an immune response in a subject comprising administering to the subject the pharmaceutical composition of claim 19.

21. The method of claim 20, wherein the subject has a tumor and an immune response against the tumor is stimulated.

22. The method of claim 20, wherein the subject has a chronic viral infection and an immune response against the viral infection is stimulated.

23. A method of treating cancer comprising administering to a subject in need thereof a therapeutically effective amount of the pharmaceutical composition of claim 19.

24. The method of claim 23, wherein the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

25. A method of treating a chronic viral infection comprising administering to a subject in need thereof a therapeutically effective amount of the pharmaceutical composition of claim 19.


SEQUENCE LISTING (186 sequences)

SEQ ID NO: 1 - protein, 277 aa, Homo sapiens
  SIGNAL (1)..(20)
  PEPTIDE (21)..(277)
  DOMAIN (21)..(193): extracellular domain
  DOMAIN (194)..(215): transmembrane domain
  DOMAIN (216)..(277): intracellular domain
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 448 aa, Artificial Sequence
  chimeric antibody with mouse variable domain, human constant domain
  DOMAIN (1)..(119): variable domain
  MISC_FEATURE (31)..(35): CDRH1
  MISC_FEATURE (50)..(66): CDRH2
  MISC_FEATURE (99)..(108): CDRH3
EVQLQQSGPELEKPGASVKMSCKASGYSFTGYNMNWVKQSNGKSLEWIGNIDPYYGNTNYNQKFKGKATLTVDKSSSTAYLQLKSLTSEDSAVYYCARLGLQLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 219 aa, Artificial Sequence
  chimeric antibody with mouse variable domain and human constant domain
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DVVMTQTPLSLPVSLGDQASISCRSSQSLVHSNGNTYLHWYLQKPGQSPNLLIYKLTNRFFGVPDRFSGSGSGTDFTLKISRVEAEDLGVYFCSQSIHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial Sequence
  humanized antibody with mouse CDRs and human framework and constant regions
  DOMAIN (1)..(119): variable domain
  MISC_FEATURE (31)..(35): CDRH1
  MISC_FEATURE (50)..(66): CDRH2
  MISC_FEATURE (99)..(108): CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQTPLSLPVTPGEPASISCRSSQSLVHSNGNTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, Artificial Sequence; identical to SEQ ID NO: 5
  humanized antibody with mouse CDRs and human framework and constant regions
  DOMAIN (1)..(119): variable domain
  MISC_FEATURE (31)..(35): CDRH1
  MISC_FEATURE (50)..(66): CDRH2
  MISC_FEATURE (99)..(108): CDRH3

SEQ ID NO: 8 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQTPLSLPVTPGEPASISCRSSQSLVHSNANTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 448 aa, Artificial Sequence
  humanized antibody with mouse CDRs and human framework and constant regions
  DOMAIN (1)..(119): variable domain
  MISC_FEATURE (31)..(35): CDRH1
  MISC_FEATURE (50)..(66): CDRH2
  MISC_FEATURE (99)..(108): CDRH3
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 219 aa, Artificial Sequence; identical to SEQ ID NO: 6
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3

SEQ ID NO: 11 - protein, 448 aa, Artificial Sequence; identical to SEQ ID NO: 9
  humanized antibody with mouse CDRs and human framework and constant regions
  DOMAIN (1)..(119): variable domain
  MISC_FEATURE (31)..(35): CDRH1
  MISC_FEATURE (50)..(66): CDRH2
  MISC_FEATURE (99)..(108): CDRH3

SEQ ID NO: 12 - protein, 218 aa, Artificial Sequence
  chimeric antibody with mouse variable domain and human constant domain
  DOMAIN (1)..(111): variable domain
  MISC_FEATURE (24)..(38): CDRL1
  MISC_FEATURE (54)..(60): CDRL2
  MISC_FEATURE (93)..(101): CDRL3
DIVLTQSPASLAVSLGQRATISCRASKNVDSYGNSFMHWYQQKPGQPPKLLIYRASNLESGIPARFGGSGSGTDFTLTINPVEADDVATYYCQQSNEDPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, Artificial Sequence
  chimeric antibody with mouse variable domain and human constant domain
  DOMAIN (1)..(117): variable domain
  MISC_FEATURE (31)..(35): CDRH1
  MISC_FEATURE (50)..(66): CDRH2
  MISC_FEATURE (99)..(106): CDRH3
QVQLQQSAAELARPGASVKMSCKASGYTFTDLSMHWIKQRPGQGLEWIGYITPSSGYTAYNQKFKGKTTLTADKSSSTAYMQLSSLTSEDSAVYYCARLILQRGAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 446 aa, Artificial Sequence
  humanized antibody with mouse CDRs and human framework and constant regions
  DOMAIN (1)..(117): variable domain
  MISC_FEATURE (31)..(35): CDRH1
  MISC_FEATURE (50)..(66): CDRH2
  MISC_FEATURE (99)..(106): CDRH3
QVQLVQSGAEVKKPGSSVKVSCKASGYTFTDLSMHWIKQRPGQGLEWIGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 218 aa, Artificial Sequence
  humanized antibody with mouse CDRs and human framework and constant regions
  DOMAIN (1)..(111): variable domain
  MISC_FEATURE (24)..(38): CDRL1
  MISC_FEATURE (54)..(60): CDRL2
  MISC_FEATURE (93)..(101): CDRL3
DIVMTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 446 aa, Artificial Sequence; identical to SEQ ID NO: 14
  humanized antibody with mouse CDRs and human framework and constant regions
  DOMAIN (1)..(117): variable domain
  MISC_FEATURE (31)..(35): CDRH1
  MISC_FEATURE (50)..(66): CDRH2
  MISC_FEATURE (99)..(106): CDRH3

SEQ ID NO: 17 - protein, 218 aa, Artificial Sequence
  humanized antibody with mouse CDRs and human framework and constant regions
  DOMAIN (1)..(111): variable domain
  MISC_FEATURE (24)..(38): CDRL1
  MISC_FEATURE (54)..(60): CDRL2
  MISC_FEATURE (93)..(101): CDRL3
DIVLTQSPDSLAVSLGERATISCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 446 aa, Artificial Sequence
  humanized antibody with mouse CDRs and human framework and constant regions
  DOMAIN (1)..(117): variable domain
  MISC_FEATURE (31)..(35): CDRH1
  MISC_FEATURE (50)..(66): CDRH2
  MISC_FEATURE (99)..(106): CDRH3
QVQLVQSGAEVKKPGSSVKVSCKASGYTFTDLSMHWVRQAPGQGLEWMGYITPSSGYTAYNQKFKGKTTLTADKSTSTAYMELSSLRSEDTAVYYCARLILQRGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 218 aa, Artificial Sequence
  humanized antibody with mouse CDRs and human framework and constant regions
  DOMAIN (1)..(111): variable domain
  MISC_FEATURE (24)..(38): CDRL1
  MISC_FEATURE (54)..(60): CDRL2
  MISC_FEATURE (93)..(101): CDRL3
DIVLTQSPDSLAVSLGERATINCRASKNVDSYGNSFMHWYQQKPGQPPKLLIYRASNLESGVPDRFSGSGSGTDFTLTISSLQAEDVAVYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, Artificial Sequence
  chimeric antibody with mouse variable domain and human constant domain
  DOMAIN (1)..(122): variable domain
  MISC_FEATURE (31)..(35): CDRH1
  MISC_FEATURE (50)..(66): CDRH2
  MISC_FEATURE (99)..(111): CDRH3
QVQFQQSGAELARPGASVKLSCKASGYTFTSYWMQWVKQRPGQGLEWIGTIYPGDGDSRYNQKFKGKALLTADKSSSIAYMQLNSLASEDSAVYFCARFSLYDGYPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 219 aa, Artificial Sequence
  chimeric antibody with mouse variable domain and human constant domain
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DVVMTQTPLSLPVSLGDQASISCRSSQSLVHRNGNTYLHWYLQKPGQSPKLLIYRVSNRFSGVPDRFSGSGSGTDFTLKISRVEAEDLGIYFCSQSTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 451 aa, Artificial Sequence
  humanized antibody with mouse CDRs and human framework and constant regions
  DOMAIN (1)..(122): variable domain
  MISC_FEATURE (31)..(35): CDRH1
  MISC_FEATURE (50)..(66): CDRH2
  MISC_FEATURE (99)..(111): CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYDGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQSPLSLPVTPGEPASISCRSSQSLVHRNGNTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 451 aa, Artificial Sequence
  humanized antibody with mouse CDRs and human framework and constant regions
  DOMAIN (1)..(122): variable domain
  MISC_FEATURE (31)..(35): CDRH1
  MISC_FEATURE (50)..(66): CDRH2
  MISC_FEATURE (99)..(111): CDRH3
QVQFVQSGAEVKKPGSSVKVSCKASGYTFTSYWMQWVRQAPGQGLEWMGTIYPGDGDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, Artificial Sequence; identical to SEQ ID NO: 23
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3

SEQ ID NO: 26 - protein, 451 aa, Artificial Sequence; identical to SEQ ID NO: 24
  humanized antibody with mouse CDRs and human framework and constant regions
  DOMAIN (1)..(122): variable domain
  MISC_FEATURE (31)..(35): CDRH1
  MISC_FEATURE (50)..(66): CDRH2
  MISC_FEATURE (99)..(111): CDRH3

SEQ ID NO: 27 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQSPLSLPVTPGEPASISCRSSQSLVHRNANTYLHWYLQKPGQSPQLLIYRVSNRFSGVPDRFSGSGSGTDFTLKISRVEAEDVGVYYCSQSTHFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 451 aa, Artificial Sequence
  humanized antibody with mouse CDRs and human framework and constant regions
  DOMAIN (1)..(122): variable domain
  MISC_FEATURE (31)..(35): CDRH1
  MISC_FEATURE (50)..(66): CDRH2
  MISC_FEATURE (99)..(111): CDRH3
QVQFVQSGAEVKKPGSSVKVSCKASGYTFTSYWMQWVRQAPGQGLEWMGTIYPGDSDSRYNQKFKGKALLTADKSTSTAYMELSSLRSEDTAVYYCARFSLYEGYPYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 219 aa, Artificial Sequence; identical to SEQ ID NO: 23
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3

SEQ ID NO: 30 - protein, 451 aa, Artificial Sequence; identical to SEQ ID NO: 28
  humanized antibody with mouse CDRs and human framework and constant regions
  DOMAIN (1)..(122): variable domain
  MISC_FEATURE (31)..(35): CDRH1
  MISC_FEATURE (50)..(66): CDRH2
  MISC_FEATURE (99)..(111): CDRH3

SEQ ID NO: 31 - protein, 219 aa, Artificial Sequence; identical to SEQ ID NO: 27
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3

SEQ ID NO: 32 - protein, 442 aa, Artificial Sequence
  chimeric antibody with mouse variable domain and human constant domain
  DOMAIN (1)..(113): variable domain
  MISC_FEATURE (31)..(35): CDRH1
  MISC_FEATURE (50)..(66): CDRH2
  MISC_FEATURE (99)..(102): CDRH3
EVLLQQSGPELVKPGASVKIPCKASGYKFTDYNMDWVKQSHGKSLEWIGDINPKNGGTIYNLKFKGKATLTVDMSSSTAYMELRSLTSEDTAVYYCVRRMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 214 aa, Artificial Sequence
  chimeric antibody with mouse variable domain and human constant domain
  DOMAIN (1)..(107): variable domain
  MISC_FEATURE (24)..(34): CDRL1
  MISC_FEATURE (50)..(56): CDRL2
  MISC_FEATURE (89)..(97): CDRL3
DIQMTQTTSSLSASLGDRVTISCRASQDISNYLNWYQQKPDGTVKLLIYYISRLHSGVPSRFRGSGSGTDFSLTISNLEQEDIATYFCQQGNTLPYTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 442 aa, Artificial Sequence
  humanized antibody with mouse CDRs and human framework and constant regions
  DOMAIN (1)..(113): variable domain
  MISC_FEATURE (31)..(35): CDRH1
  MISC_FEATURE (50)..(66): CDRH2
  MISC_FEATURE (99)..(102): CDRH3
QVQLVQSGAEVKKPGASVKVSCKASGYTFTDYNMDWVRQAPGQGLEWMGDINPKNGGTIYNLKFKGRVTLTTDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 214 aa, Artificial Sequence
  humanized antibody with mouse CDRs and human framework and constant regions
  DOMAIN (1)..(107): variable domain
  MISC_FEATURE (24)..(34): CDRL1
  MISC_FEATURE (50)..(56): CDRL2
  MISC_FEATURE (89)..(97): CDRL3
DIQMTQSPSSLSASVGDRVTITCRASQDISNYLNWYQQKPGKVPKLLIYYISRLHSGVPSRFSGSGSGTDFTLTISSLQPEDVATYYCQQGNTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 442 aa, Artificial Sequence
  humanized antibody with mouse CDRs and human framework and constant regions
  DOMAIN (1)..(113): variable domain
  MISC_FEATURE (31)..(35): CDRH1
  MISC_FEATURE (50)..(66): CDRH2
  MISC_FEATURE (99)..(102): CDRH3
QVQLVQSGAEVKKPGASVKVSCKASGYTFTDYNMDWVRQAPGQGLEWMGDINPKNAGTIYNLKFKGRVTLTVDTSTSTAYMELRSLRSDDTAVYYCVRRMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 214 aa, Artificial Sequence; identical to SEQ ID NO: 35
  humanized antibody with mouse CDRs and human framework and constant regions
  DOMAIN (1)..(107): variable domain
  MISC_FEATURE (24)..(34): CDRL1
  MISC_FEATURE (50)..(56): CDRL2
  MISC_FEATURE (89)..(97): CDRL3

SEQ ID NO: 38 - protein, 441 aa, Artificial Sequence
  chimeric antibody with mouse variable domain and human constant domain
  DOMAIN (1)..(112): variable domain
  MISC_FEATURE (31)..(35): CDRH1
  MISC_FEATURE (50)..(66): CDRH2
  MISC_FEATURE (99)..(101): CDRH3
EVQLVESGGGLVKPGGSLKLSCAASGFTFSSYAMSWVRQSPEKRLEWVAEISGGGSYTYYPDTVTGRFTISRDNAKNTLYLEMSSLRSEDTAMYYCASRAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 219 aa, Artificial Sequence
  chimeric antibody with mouse variable domain and human constant domain
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DVLMTQTPLSLPVSLGDQASISCRSSQSIVHSNGNTYFEWYLQKPGQSPKLLIYKVSNRFSGVPDRFSGSGSGTDFTLKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 441 aa, Artificial Sequence
  humanized antibody with mouse CDRs and human framework and constant regions
  DOMAIN (1)..(112): variable domain
  MISC_FEATURE (31)..(35): CDRH1
  MISC_FEATURE (50)..(66): CDRH2
  MISC_FEATURE (99)..(101): CDRH3
EVQLVESGGGLVKPGGSLRLSCAASGFTFSSYAMSWVRQAPGKGLEWVSEISGGGSYTYYPDTVTGRFTISRDNAKNSLYLQMNSLRAEDTAVYYCASRAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQTPLSLPVTPGEPASISCRSSQSIVHSNGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 441 aa, Artificial Sequence; identical to SEQ ID NO: 40
  humanized antibody with mouse CDRs and human framework and constant regions
  DOMAIN (1)..(112): variable domain
  MISC_FEATURE (31)..(35): CDRH1
  MISC_FEATURE (50)..(66): CDRH2
  MISC_FEATURE (99)..(101): CDRH3

SEQ ID NO: 43 - protein, 219 aa, Artificial Sequence
  humanized antibody with mouse CDRs and human framework and constant regions
  DOMAIN (1)..(112): variable domain
  MISC_FEATURE (24)..(39): CDRL1
  MISC_FEATURE (55)..(61): CDRL2
  MISC_FEATURE (94)..(102): CDRL3
DIVMTQTPLSLPVTPGEPASISCRSSQSIVHSQGNTYFEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 107 aa, Homo sapiens [sequence omitted]

SEQ ID NO: 46 - protein, 17 aa, Homo sapiens
MRAWIFFLLCLAGRALA

SEQ ID NO: 47 - protein, 5 aa, Homo sapiens
VDKRV

SEQ ID NO: 48 - protein, 10 aa, Homo sapiens
EPKSCDKTHT

SEQ ID NO: 49 - protein, 5 aa, Homo sapiens
CPPCP

SEQ ID NO: 50 - protein, 7 aa, Homo sapiens
APELLGG

SEQ ID NO: 51 - protein, 5 aa, Homo sapiens
VDKTV

SEQ ID NO: 52 - protein, 9 aa, Homo sapiens
CCVECPPCP

SEQ ID NO: 53 - protein, 6 aa, Homo sapiens
APPVAG

SEQ ID NO: 54 - protein, 12 aa, Homo sapiens
ELKTPLGDTTHT

SEQ ID NO: 55 - protein, 50 aa, Homo sapiens
CPRCPEPKSCDTPPPCPRCPEPKSCDTPPPCPRCPEPKSCDTPPPCPRCP

SEQ ID NO: 56 - protein, 35 aa, Homo sapiens
CPRCPEPKSCDTPPPCPRCPEPKSCDTPPPCPRCP

SEQ ID NO: 57 - protein, 20 aa, Homo sapiens
CPRCPEPKSCDTPPPCPRCP

SEQ ID NO: 58 - protein, 4 aa, Homo sapiens
EPKS

SEQ ID NO: 59 - protein, 41 aa, Homo sapiens
CDTPPPCPRCPEPKSCDTPPPCPRCPEPKSCDTPPPCPRCP

SEQ ID NO: 60 - protein, 11 aa, Homo sapiens
CDTPPPCPRCP

SEQ ID NO: 61 - protein, 7 aa, Homo sapiens
ESKYGPP

SEQ ID NO: 62 - protein, 5 aa, Homo sapiens
CPSCP

SEQ ID NO: 63 - protein, 7 aa, Homo sapiens
APEFLGG

SEQ ID NO: 64 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 65 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 66 - protein, 103 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 67 - protein, 103 aa, Artificial Sequence
  human sequence with A330S and P331S mutations
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAK

SEQ ID NO: 68 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 326 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 325 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 325 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRREMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 325 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 325 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 325 aa, Artificial Sequence; identical to SEQ ID NO: 70
  human sequences from different isotypes with C219S mutation

SEQ ID NO: 75 - protein, 325 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 325 aa, Artificial Sequence
  human sequences from different isotypes with C219S mutation
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 18 aa, Homo sapiens
ERKCCVECPPCPAPPVAG

SEQ ID NO: 78 - protein, 18 aa, Artificial Sequence
  human sequence with C219S mutation
ERKSCVECPPCPAPPVAG

SEQ ID NO: 79 - protein, 19 aa, Artificial Sequence
  human sequences from different isotypes
ERKCCVECPPCPAPELLGG

SEQ ID NO: 80 - protein, 19 aa, Artificial Sequence
  human sequences from different isotypes with C219S mutation
ERKSCVECPPCPAPELLGG

SEQ ID NO: 81 - protein, 22 aa, Homo sapiens
EPKSCDKTHTCPPCPAPELLGG

SEQ ID NO: 82 - protein, 22 aa, Artificial Sequence
  human sequence with L234A, L235E and G237A mutations
EPKSCDKTHTCPPCPAPEAEGA

SEQ ID NO: 83 - protein, 103 aa, Homo sapiens
PSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 84 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 87 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 326 aa, Homo sapiens
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 90 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 327 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 329 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVETHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 94 - protein, 329 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVEGGGCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 95 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 327 aa, Homo sapiens
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 326 aa, Homo sapiens
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 329 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVETHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - protein, 327 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVERKSCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 326 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 330 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 330 aa, Artificial Sequence
  human sequences from different isotypes
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 326 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 104 - protein, 330 aa, Artificial Sequence
  human sequence with artificial mutations
ASTKGPSVFPLAPSSRSTSESTAALGCLVKDYFPEPVTVSWNSGALTSGVHTFPAVLQSSGLYSLSSVVTVPSSNFGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 330 aa, Artificial Sequence
  human sequence with artificial mutations
ASTKGPSVFPLAPSSRSTSESTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 330 aa, Artificial Sequence
  human sequence with artificial mutations
ASTKGPSVFPLAPSSKSTSGGTAALGCLVKDYFPEPVTVSWNSGALTSGVHTFPAVLQSSGLYSLSSVVTVPSSNFGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 330 aa, Artificial Sequence
  human sequence with artificial mutations
ASTKGPSVFPLAPSSKSTSGGTAALGCLVKDYFPEPVTVSWNSGALTSGVHTFPAVLQSSGLYSLSSVVTVPSSSLGTQTYTCNVDHKPSNTKVDKTVERKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 329 aa, Artificial Sequence
  human sequence with artificial mutations
ASTKGPSVFPLAPSSKSTSGGTAALGCLVKDYFPEPVTVSWNSGALTSGVHTFPAVLQSSGLYSLSSVVTVPSSNFGTQTYICNVNHKPSNTKVDKRVERKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 109 - protein, 326 aa, Artificial Sequence
  human sequence with artificial mutations
ASTKGPSVFPLAPCSKSTSGGTAALGCLVKDYFPEPVTVSWNSGALTSGVHTFPAVLQSSGLYSLSSVVTVPSSSLGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 110 - protein, 326 aa, Artificial Sequence
  human sequence with artificial mutations
ASTKGPSVFPLAPCSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 111 - protein, 326 aa, Artificial Sequence
  human sequence with artificial mutations
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 112 - protein, 326 aa, Artificial Sequence
  human sequence with artificial mutations
ASTKGPSVFPLAPCSRSTSESTAALGCLVKDYFPEPVTVSWNSGALTSGVHTFPAVLQSSGLYSLSSVVTVPSSNFGTQTYICNVNHKPSNTKVDKRVEPKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 113 - protein, 326 aa, Artificial Sequence
  human sequence with artificial mutations
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVEPKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 114 - protein, 330 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 329 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 326 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - protein, 326 aa, Artificial Sequence
  human sequences from different isotypes
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 326 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 119 - protein, 327 aa, Artificial Sequence
  human sequences from different isotypes
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 120 - protein, 326 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 330 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 122 - protein, 330 aa, Artificial Sequence
  human sequences from different isotypes
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 123 - protein, 327 aa, Artificial Sequence
  human sequences from different isotypes
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 328 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 125 - protein, 329 aa, Artificial Sequence
  human sequences from different isotypes
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 126 - protein, 323 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 322 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 128 - protein, 322 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 129 - protein, 328 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 327 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 131 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 132 - protein, 326 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 133 - protein, 18 aa, Artificial Sequence
  human sequence with C220S mutation
ERKCSVECPPCPAPPVAG

SEQ ID NO: 134 - protein, 19 aa, Artificial Sequence
  human sequences from different isotypes and C220S mutation
ERKCSVECPPCPAPELLGG

SEQ ID NO: 135 - protein, 14 aa, Homo sapiens
ERKCCVECPPCPAP

SEQ ID NO: 136 - protein, 14 aa, Artificial Sequence
  human sequence with C219S mutation
ERKSCVECPPCPAP

SEQ ID NO: 137 - protein, 14 aa, Artificial Sequence
  human sequence with C220S mutation
ERKCSVECPPCPAP

SEQ ID NO: 138 - protein, 14 aa, Artificial Sequence
  human sequence with C219X mutation; X = any amino acid other than cysteine
  Xaa (4)..(4): Xaa = any amino acid other than cysteine
ERKXCVECPPCPAP

SEQ ID NO: 139 - protein, 14 aa, Artificial Sequence
  human sequence with C220X mutation
  Xaa (5)..(5): Xaa = any amino acid other than cysteine
ERKCXVECPPCPAP

SEQ ID NO: 140 - protein, 116 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAG

SEQ ID NO: 141 - protein, 18 aa, Artificial Sequence
  human sequence with C219X mutation
  misc_feature (4)..(4): Xaa can be any naturally occurring amino acid
  Xaa (5)..(5): Xaa = any amino acid other than cysteine
ERKXCVECPPCPAPPVAG

SEQ ID NO: 142 - protein, 18 aa, Artificial Sequence
  human sequence with C220X mutation
  Xaa (5)..(5): Xaa = any amino acid other than cysteine
ERKCXVECPPCPAPPVAG

SEQ ID NO: 143 - protein, 19 aa, Artificial Sequence
  human sequences from different isotypes and C219X mutation
  Xaa (4)..(4): Xaa = any amino acid other than cysteine
ERKXCVECPPCPAPELLGG

SEQ ID NO: 144 - protein, 19 aa, Artificial Sequence
  human sequences from different isotypes and C220X mutation
  Xaa (5)..(5): Xaa = any amino acid other than cysteine
ERKCXVECPPCPAPELLGG

SEQ ID NO: 145 - protein, 18 aa, Artificial Sequence
  human sequences from different isotypes and G deletion
ERKCCVECPPCPAPELLG

SEQ ID NO: 146 - protein, 18 aa, Artificial Sequence
  human sequence from different isotypes, with C219S mutation and G deletion
ERKSCVECPPCPAPELLG

SEQ ID NO: 147 - protein, 18 aa, Artificial Sequence
  human sequence from different isotypes, with C220S mutation and G deletion
ERKCSVECPPCPAPELLG

SEQ ID NO: 148 - protein, 18 aa, Artificial Sequence
  human sequence from different isotypes, with C219X mutation and G deletion
  Xaa (4)..(4): Xaa = any amino acid other than cysteine
ERKXCVECPPCPAPELLG

SEQ ID NO: 149 - protein, 18 aa, Artificial Sequence
  human sequence from different isotypes, with C220X mutation and G deletion
  Xaa (5)..(5): Xaa = any amino acid other than cysteine
ERKCXVECPPCPAPELLG

SEQ ID NO: 150 - protein, 4 aa, Homo sapiens
PVAG

SEQ ID NO: 151 - protein, 7 aa, Homo sapiens
SCDKTHT

SEQ ID NO: 152 - protein, 4 aa, Homo sapiens
ELLG

SEQ ID NO: 153 - protein, 5 aa, Homo sapiens
ELLGG

SEQ ID NO: 154 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 326 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 160 - protein, 327 aa, Artificial Sequence
  human sequences from different isotypes with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 327 aa, Artificial Sequence
  human sequences from different isotypes
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 327 aa, Artificial Sequence
  human sequences from different isotypes with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEAEGAKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 327 aa, Artificial Sequence
  human sequences from different isotypes with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 327 aa, Artificial Sequence
  human sequences from different isotypes with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 166 - protein, 327 aa, Artificial Sequence
  human sequences from different isotypes with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 327 aa, Artificial Sequence
  human sequences from different isotypes with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 327 aa, Artificial Sequence
  human sequences from different isotypes with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEAEGAKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 327 aa, Artificial Sequence
  human sequences from different isotypes with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 327 aa, Artificial Sequence
  human sequences from different isotypes with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELEGGKSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 174 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 177 - protein, 330 aa, Artificial Sequence
  human sequence with artificial mutation(s)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 326 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRKPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 179 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRKPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 180 - protein, 326 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 181 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 182 - protein, 326 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKYLSLSPGK

SEQ ID NO: 183 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKYLSLSPGK

SEQ ID NO: 184 - protein, 326 aa, Artificial Sequence
  human sequence with artificial mutation(s)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 185 - protein, 326 aa, Artificial Sequence
  human sequence with artificial mutation(s)
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 186 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK